(19) United States
(12) Patent Application Publication
(10) Pub. No.: US 2026/0345795 A1
(43) Pub. Date: Nov. 5, 2026

(54) MULTIVALENCY FOR ENZYME INHIBITION

(71) Applicant: Indiana University Research and Technology Corporation, Bloomington, IN (US)

(72) Inventors: Nathan Joseph ALVES, Zionsville, IN (US); Tanmaye NALLAN CHAKRAVARTHULA, Indianapolis, IN (US)

(21) Appl. No.: 19/109,875

(22) PCT Filed: Aug. 28, 2023
(86) PCT No.: PCT/US2023/031299

§ 371 Date: Mar. 7, 2025

Related U.S. Application Data
(60) Provisional application No. 63/405,172, filed on Sep. 9, 2022.

(51) Int. Cl. A61K 47/60; A61K 47/55; A61P 7/04

(52) U.S. Cl. CPC A61K 47/60; A61K 47/55; A61P 7/04

(57) ABSTRACT

Disclosed are compositions, uses of the compositions, methods of treatment, methods of modulating enzyme activity, methods of sequestering a target molecule, methods of delivering an enzyme, and methods of prophylaxis. In particular, the present disclosure is directed to multivalent affinity molecules having an affinity moiety and a scaffold. Multiple affinity moieties share a common scaffold to increase valency and reduce entropic penalty to produce multivalent affinity molecules with stronger and/or selective inhibition of an enzyme, for example. The multivalent affinity molecules of the present disclosure are particularly suitable for prophylaxis and treating bleeding and thrombosis associated disorders in a subject in need thereof, for modulating enzyme activity, for sequestering enzymes, and for delivering enzymes.

### CROSS-REFERENCE TO RELATED APPLICATIONS

This application claims priority under 35 U.S.C. § 119(e) to U.S. Provisional Patent Application Ser. No. 63/405,172, filed on Sep. 9, 2022, the disclosure of which is hereby incorporated by reference in its entirety.

### BACKGROUND

The present disclosure relates generally to compositions, methods for treatment, methods for delivery, methods of sequestering, and methods for enhancing or reducing enzyme activity. In particular, the present disclosure is directed to multivalent affinity molecules that portray multivalent effect by having affinity moieties and a shared scaffold formed by linker moieties. Multivalent affinity molecules of the present disclosure achieve stronger and/or selective binding to target molecules such as enzymes utilizing mechanisms of multivalent binding. Stronger and/or selective binding to a target molecule can be achieved either by increasing valency and/or decreasing distance (linker length) between affinity moieties to reduce entropic penalty or by targeting multiple binding sites on the target molecule. The multivalent affinity molecules of the present disclosure are particularly suitable for treating bleeding disorders and thrombosis associated disorders in subjects in need thereof.

Multivalency (multivalent avidity) is a prevalent mechanism in nature used to achieve strong and selective, yet reversible, interactions It is defined as the enhanced response observed with multiple binding ligands linked on a common scaffold compared to the total response observed with equivalent number of monovalent ligands. To achieve similar inhibition and/or binding of target molecules such as enzymes, large amounts of monovalent ligands or ligands with very high affinity must be produced. Velcro is a common synthetic material that macroscopically portrays multivalency in which numerous weak loop and hook interactions result in an overall strong association between two surfaces. Interactions between E. coli and urethral endothelial cells, transcription factors and DNA, hemagglutinin (HA) on influenza virus, and sialic acid (SA) on bronchial epithelial cells are all naturally occurring examples of multivalency.

Active site plasmin inhibitors are useful for treating hyperfibrinolysis-associated bleeding disorders as well as cancer and inflammatory disorders caused by excessive plasmin activity. For instance, Tranexamic acid (TXA) and ε-Aminocaproic Acid (EACA) are lysine analogues that are currently FDA approved antifibrinolytic agents. These are primarily non-competitive inhibitors that bind to kringle domains of plasmin(ogen) and block plasmin(ogen)-fibrin interactions. Although, TXA is 10-fold more potent than EACA, large clinical trials have shown TXA to be ineffective or harmful if treatment is delayed beyond 3 hours. However, some drugs can be non-specific to plasmin and inhibit other serine proteases with similar active sites. Accordingly, there exists a need to provide alternative affinity molecules having improved inhibition and/or more selective binding. There also exists a need to develop alternative compositions that are strong and selective plasmin inhibitors for treating hyperfibrinolysis-associated bleeding events and for thrombin inhibitors that can prevent and treat thrombosis associated disorders such as deep vein thrombosis (DVT), ischemic stroke, and myocardial infarction caused by pathological clotting in veins and arteries. The present disclosure satisfies this need by leveraging multivalency (multivalent avidity) to eliminate the need for producing large amounts of monovalent ligands or monovalent ligands with very high affinity. Multivalent affinity molecules of the present disclosure have multiple affinity moieties that share a common scaffold formed by linker moieties. Exemplary multivalent affinity molecules of the present disclosure targeting serine protease inhibition demonstrate enhanced and selective inhibition. This multivalent approach is also applicable to any enzyme.

### BRIEF DESCRIPTION OF THE DISCLOSURE

The present disclosure relates generally to compositions and methods for treatment and prevention of thrombosis and bleeding disorders. In particular, the present disclosure is directed to multivalent affinity molecules having multiple affinity moieties and sharing a common scaffold formed by linker moieties. Multivalent affinity molecules of the present disclosure having multiple affinity moieties share a common linker scaffold to achieve stronger and/or selective binding utilizing mechanisms of multivalency. Stronger and selective inhibition of a target enzyme can be achieved either by increasing valency and/or decreasing linker length to reduce entropic penalty or by targeting multiple binding sites on an enzyme. The multivalent binding molecules of the present disclosure are particularly suitable for treating bleeding disorders and thrombosis associated disorders in subjects in need thereof. Bleeding disorders can also be treated by delivering enzymes such as thrombin that aid in coagulation using these multivalent inhibitor systems.

In one aspect, the present disclosure is directed to a multivalent affinity molecule comprising: two affinity moieties to about 100 affinity moieties; and a scaffold, wherein the two affinity moieties to about 100 affinity moieties are covalently coupled to the scaffold.

In one aspect, the present disclosure is directed to a method of treating a hyper-fibrinolysis associated bleeding disorder in a subject in need thereof, the method comprising: administering to the subject in need thereof a multivalent affinity molecule comprising two affinity moieties to about 100 affinity moieties covalently coupled to a scaffold, wherein the affinity moiety is an inhibitor of enzymes that promote fibrinolysis (such as plasmin(ogen) and tPA) and are selected from benzamidine, benzylamine, tranexamic acid, lysine, ε-aminocaproic acid, and combinations thereof. They can also be reactive cyclohexanones, nitrile warheads, aldehyde peptidomimetics, cyclic peptidomimetics, polypeptides, non-naturally occurring synthetic haptens, polypeptides of the Kunitz and Kazal-type, and lysine analogs.

In one aspect, the present disclosure is directed to a method of treating a thrombosis associated disorder in a subject in need thereof, the method comprising: administering to the subject in need thereof a multivalent affinity molecule comprising: two affinity moieties to about 100 affinity moieties covalently coupled to a scaffold wherein the affinity moiety is selected from benzamidine derivatives, peptidomimetics, arginomimetic, orcinol-based, aryl-based, phenylguanidine, argatroban, gabexate mesylate, nafamostat mesylate, efegatran, inogatran and napsagatran, melagatran, ximelagatran, serine protease haptens, kringle domain binders and combinations thereof. Thrombosis-associated disorders can also be treated by delivering enzymes that aid in fibrinolysis such as plasmin and tPA using these multivalent inhibitor systems.

In one aspect, the present disclosure is directed to a method of modulating an enzyme, the method comprising: contacting the enzyme with a multivalent affinity molecule comprising: two affinity moieties to about 100 affinity moieties that specifically bind the enzyme; and a scaffold, wherein the two affinity moieties to about 100 affinity moieties are covalently coupled to the scaffold and wherein upon binding of at least one of the two affinity moieties to about 100 affinity ligands to the enzyme modulates the activity of the enzyme.

In one aspect, the present disclosure is directed to a method of sequestering an enzyme, the method comprising: contacting the enzyme with a multivalent affinity molecule, the multivalent affinity molecule comprising: two affinity moieties to about 100 affinity moieties that specifically bind the enzyme; and a scaffold, wherein the two affinity moieties to about 100 affinity moieties are covalently coupled to the scaffold and wherein at least one of the two affinity moieties to about 100 affinity moieties are reversibly coupled to the enzyme to sequester the enzyme.

In one aspect, the present disclosure is directed to a method of delivering an enzyme, the method comprising: contacting the enzyme with a multivalent affinity molecule, the multivalent affinity molecule comprising: two affinity moieties to about 100 affinity moieties that reversibly bind the enzyme; and a scaffold, wherein the two affinity moieties to about 100 affinity moieties are covalently coupled to the scaffold and wherein the two affinity moieties to about 100 affinity moieties are reversibly coupled to the enzyme to form an enzyme-multivalent affinity molecule complex; providing the enzyme-multivalent affinity molecule complex to a target site; wherein the enzyme is released from the enzyme-multivalent affinity molecule complex at the target site to deliver the enzyme.

In one aspect, the present disclosure is directed to a method of prophylaxis of a bleeding disorder in a subject in need thereof, the method comprising: administering to the subject in need thereof a multivalent affinity molecule comprising: two affinity moieties to about 100 affinity moieties covalently coupled to a scaffold.

In one aspect, the present disclosure is directed to a method of prophylaxis of thrombosis in a subject in need thereof, the method comprising: administering to the subject in need thereof a multivalent affinity molecule comprising: two affinity moieties to about 100 affinity moieties covalently coupled to a scaffold.

In accordance with the present disclosure, compositions and methods have been discovered that portray multivalent effect and surprisingly allow for treating and/or prophylaxis of bleeding disorders or thrombosis associated disorders in a subject in need thereof. The multivalent affinity molecules of the present disclosure having multiple affinity moieties that share a common scaffold to increase valency and lower linker length by reducing entropic penalty and/or target multiple binding sites to produce enzyme inhibition with stronger and/or selective inhibition of a target enzyme.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the depicts the mechanisms of multivalency: Statistical rebinding; Chelation; Subsite binding; and Clustering. Enzyme inhibitors are represented in blue whereas as different enzymes are represented in various colors.

FIG. 2 depicts the influence of varying valency and linker length on the multivalent inhibition of plasmin by benzamidine via statistical rebinding.

FIG. 3 is a schematic illustration depicting statistical rebinding of homo-bivalent benzamidine to the active site of plasmin and subsite rebinding of hetero-bivalent benzamidine-TXA showing benzamidine binding to the plasmin active site and TXA binding to the plasmin lysine binding sites (LBS). The actual Kringle domain binding location depends upon linker length.

FIGS. 4A-4C depict a schematic for synthesis of monovalent, bivalent and trivalent benzamidine (FIG. 4A) and exemplary data for synthesized Bis-dPEG2-AMB: HPLC Chromatogram (FIG. 4B) and Mass Spectrum (FIG. 4C).

FIG. 5 depicts monovalent (m-dPEGx-AMB), bivalent (Bis-dPEGx-AMB) and trivalent benzamidine (Tri-dPEGx-AMB) inhibitors shown to scale relative to plasminogen (PDB ID: 4DUR).

FIGS. 6A-6C depict a Dixon plot for Bis-dPEG2-AMB (FIG. 4A). Ki is the magnitude of negative x intersection (Ki=55.3±5.3 μM) (FIG. 6A). Comish-Bowden (SNo vs I) plot for Bis-dPEG2-AMB (FIG. 6B). Parallel lines indicate competitive inhibition. Ki vs Length for all inhibitors (FIG. 6C). Greater Ki (weaker inhibition) for longer separation lengths between benzamidines. For a specific length, Ki of monovalent (circles)>bivalent (squares)>trivalent (triangles) indicating trivalent exhibits strongest inhibition followed by bivalent and then monovalent.

FIGS. 7A and 7B depict the chemical structure (FIG. 7A) and characterization (FIG. 7B) of m-dPEG2-AMB. m-dPEG2-AMB was synthesized by reacting m-dPEG2-NHS (1 eq., 40 μmol) with AMB (3 eq., 120 μmol) in 0.01M PBS at pH 7.4 for 30 minutes at room temperature. NaOH was added as necessary to maintain pH. RP-HPLC: m-dPEG2-AMB was purified using semi-preparative Thermo Hypersil GOLD C18 column (5 um, 250×10 mm) on 5 minutes 10-15% Sol B gradient. The purified product was rotate evaporated, and the yield was 36% (4.1 mg). Analytical HPLC: Purity 83%. Retention time (Rt)=2.6 min. MS QTOF (ESI+, 220V): Mass found: 280.1849 [M+H]+; Calculated 280.1673 [M+H]+.

FIGS. 8A and 8B. depict the chemical structure (FIG. 8A) and characterization (FIG. 8B) of m-dPEG4-AMB. m-dPEG4-AMB was synthesized by reacting m-dPEG4-NHS (1 eq., 30 μmol) with AMB (3 eq., 90 μmol) in 0.01M PBS at pH 7.4 for 30 mins at room temperature. NaOH was added as necessary to maintain pH. RP-HPLC: m-dPEG4-AMB was purified using semi-preparative Thermo Hypersil GOLD C18 column (5 um, 250×10 mm) on 5 minutes 10-25% Sol B gradient. The purified product was rotate evaporated, and the yield was 40% (4.4 mg). Analytical HPLC: Purity 84% (Rt=3.1 min). MS QTOF (ESI+, 220V): Mass found: 368.2353 [M+H]⁺; Calculated 368.2173 [M+H]⁺.

FIGS. 9A and 9B depict the chemical structure (FIG. 9A) and characterization (FIG. 9B) of m-dPEG12-AMB. m-dPEG12-AMB was synthesized by reacting m-dPEG12-NHS (1 eq., 15 μmol) with AMB (3 eq., 45 μmol) in 0.01M PBS at pH 7.4 for 30 mins at room temperature. NaOH was added as necessary to maintain pH. RP-HPLC: m-dPEG12-AMB was purified using semi-preparative Thermo Hypersil GOLD C18 column (5 um, 250×10 mm) on 5 minutes 10-35% Sol B gradient. The purified product was rotate evaporated, and the yield was 43% (4.0 mg). Analytical HPLC: Purity 82% (Rt=4.1 min). MS QTOF (ESI+, 220V): Mass found: 720.3914 [M+H]⁺, 371.6794 [M+H+Na]²⁺; Calculated 720.4273 [M+H]⁺, 371.7082 [M+H+Na]²⁺.

FIGS. 10A and 10B depict the chemical structure (FIG. 10A) and characterization (FIG. 10B) of m-dPEG24-AMB. m-dPEG24-AMB was synthesized by reacting m-dPEG24-NHS (1 eq., 6 μmol) with AMB (4 eq., 24 μmol) in 0.01M PBS at pH 7.4 for 30 mins at room temperature. NaOH was added as necessary to maintain pH. RP-HPLC: m-dPEG24-AMB was purified using semi-preparative Thermo Hypersil GOLD C18 column (5 μm, 250×10 mm) on 5 minutes 10-35% Sol B gradient. The purified product was rotate evaporated, and the yield was 39% (3.0 mg). Analytical HPLC: Purity 96% (Rt=4.6 min). MS QTOF (ESI+, 220V): Mass found: 1248.7481[M+H]⁺; Calculated 1248.7473 [M+H]⁺.

FIGS. 11A and 11B depict the chemical structure (FIG. 11A) and characterization (FIG. 11B) of Bis-dPEG2-AMB. Bis-dPEG2-AMB was synthesized by reacting Bis-dPEG2-NHS (1 eq., 50 μmol) with AMB (4 eq., 200 μmol) in 0.01M PBS at pH 7.4 for 30 mins at room temperature. NaOH was added as necessary to maintain pH. RP-HPLC: Bis-dPEG2-AMB was purified using Thermo Hypersil GOLD C18 column (5 um, 250×4.6 mm) on 5 minutes 10-17% Sol B gradient. The purified product was rotate evaporated, and the yield was 53% (12.4 mg). Analytical HPLC: Purity 88% (Rt=2.5 min). MS QTOF (ESI+, 220V): Mass found: 469.2265 [M+H]⁺; Calculated 469.25 [M+H]⁺.

FIGS. 12A and 12B depict the chemical structure (FIG. 12A) and characterization (FIG. 12B) of Bis-dPEG5-AMB. Bis-dPEG5-AMB was synthesized by reacting Bis-dPEG5-NHS (1 eq., 37.5 μmol) with AMB (4 eq., 150 μmol) in 0.01M PBS at pH 7.4 for 30 mins at room temperature. NaOH was added as necessary to maintain pH. RP-HPLC: Bis-dPEG5-AMB was purified using semi-preparative Thermo Hypersil GOLD C18 column (5 μm, 250×10 mm) on 5 minutes 10-25% Sol B gradient. The purified product was rotate evaporated, and the yield was 22% (5.0 mg). Analytical HPLC: Purity 95% (Rt=3.0 min). MS QTOF (ESI+, 220V): Mass found: 601.2554 [M+H]⁺, 623.2287 [M+Na]⁺, 301.1236 [M+2H]²⁺; Calculated 601.33 [M+H]⁺, 623.33 [M+Na]⁺, 301.165 [M+2H]²⁺.

FIGS. 13A and 13B depict the chemical structure (FIG. 13A) and characterization (FIG. 13B) of Bis-dPEG13-AMB. Bis-dPEG13-AMB was synthesized by reacting Bis-dPEG13-NHS (1 eq., 22.5 μmol) with AMB (4 eq., 90 μmol) in 0.01M PBS at pH 7.4 for 30 mins at room temperature. NaOH was added as necessary to maintain pH. RP-HPLC: Bis-dPEG13-AMB was purified using Thermo Hypersil GOLD C18 column (5 μm, 250×4.6 mm) on 3.5 minutes 10-41.5% Sol B gradient. The purified product was rotate evaporated, and the yield was 48% (10.4 mg). Analytical HPLC: Purity 92% (Rt=3.7 min). MS QTOF (ESI+): Mass found: 953.3523 [M+H]⁺, 975.3211 [M+Na]⁺, 477.1843 [M+2H]²⁺; Calculated 953.54 [M+H]⁺, 975.54 [M+Na]⁺, 477.27 [M+2H]²⁺.

FIGS. 14A and 14B depict the chemical structure (FIG. 14A) and characterization (FIG. 14B) of Bis-dPEG25-AMB. Bis-dPEG25-AMB was synthesized by reacting Bis-dPEG25-NHS (1 eq., 17.5 μmol) with AMB (4 eq, 70 μmol) in 0.01M PBS at pH 7.4 for 30 mins at room temperature. NaOH was added as necessary to maintain pH. RP-HPLC: Bis-dPEG25-AMB was purified using Thermo Hypersil GOLD C18 column (5 um, 250×4.6 mm) on 8.5 minutes 10-23% Sol B gradient. The purified product was rotate evaporated, and the yield was 68% (17.8 mg). Analytical HPLC: Purity 94% (Rt=4.2 min). MS QTOF (ESI+): Mass found: 1481.8715 [M+H]+, 1503.8488 [M+Na]+, 741.4410 [M+2H]2+; Calculated 1481.85 [M+H]+, 1503.85 [M+Na]+, 741.425 [M+2H]²⁺.

FIGS. 15A and 15B depict the chemical structure (FIG. 15A) and characterization (FIG. 15B) of Tri-AMB. Tri-AMB was synthesized by reacting TSAT: Tris-(succinimidyl)aminotriacetate in DMF (1 eq., 20 μmol) with AMB in 0.01M PBS at pH 7.4 (12 eq, 240 μmol) for 30 mins while stirring at room temperature. NaOH was added as necessary to maintain pH. RP-HPLC: Tri-AMB was purified on Agilent Zorbax SB-C3 column (5 um, 250×9.4 mm) on 10 minutes 10-25% Sol B2 gradient. The purified product was rotate evaporated, and the yield was 6.4% (1.3 mg). Analytical HPLC: Purity 88% (Rt=2.4 min). MS QTOF (ESI+): Mass found: 585.3040 [M+H]⁺; Calculated 585.30 [M+H]⁺.

FIGS. 16A-16C depict the chemical structure (FIGS. 16A and 16B) and characterization (FIG. 16C) of Tri-dPEG4-AMB. Step 1—To synthesize Tri-dPEG4-AMB, first NH₂-dPEG4-AMB was synthesized. For this, Fmoc-dPEG4-NHS (1 eq., 42 gmol) was dissolved in DMF, Triethylamine (TEA) was added to it and then reacted with AMB in 0.01M PBS at pH 7.4 (3 eq, 128 μmol) for 1 hour while stirring at room temperature. The reaction crude was rotate evaporated and Fmoc was deprotected using ~40% piperidine in DMF. The resulting NH₂-dPEG4-AMB was purified and its mass was confirmed (FIG. 16A). Step 2—NH₂-dPEG4-AMB (6 eq., 42 μmol) was then reacted with TSAT (1 eq., 7 μmol) in DMF at room temperature for 15 mins after adding TEA (FIG. 16B). RP-HPLC: Tri-dPEG4-AMB was purified using semi-preparative Thermo Hypersil GOLD C18 column (5 μm, 250×10 mm) on 10 minutes 5-12.5% Sol B gradient. The purified product was rotate evaporated, and the yield was 30% (1.5 mg). Analytical HPLC: Purity 95% (Rt=3.3 min). MS QTOF (ESI+): Mass found: 1326.7501 [M+H]⁺, 1348.7331 [M+Na]+, 663.8792 [M+2H]²⁺, 685.8656 [M+2Na]²⁺, 442.9238 [M+3H]³⁺; Calculated 1326.7273 [M+H]+, 1348.7092 [M+Na]+, 663.8673 [M+2H]²⁺, 685.8492 [M+2Na]²⁺, 442.9139 [M+3H]³⁺ (FIG. 16C).

FIGS. 17A and 17B depict the chemical structure (FIG. 17A) and characterization (FIG. 7) of Tri-dPEG8-AMB. To synthesize Tri-dPEG8-AMB, first NH2-dPEG8-AMB was synthesized. Fmoc-dPEG8-NHS (1 eq., 20 μmol) was dissolved in DMF, TEA was added and then reacted with AMB in 0.01M PBS at pH 7.4 (3 eq, 60 μmol) for 1 hour while stirring at room temperature. The reaction crude was rotate evaporated and Fmoc was deprotected using ~40% piperidine in DMF. The resulting NH2-dPEG8-AMB was purified and its mass was confirmed. NH2-dPEG8-AMB (6 eq., 20 μmol) was then reacted with TSAT (1 eq., 3.3 μmol) in DMF at room temperature for 15 mins after adding TEA. RP-HPLC: Tri-dPEG8-AMB was purified using semi-preparative Thermo Hypersil GOLD C18 column (5 um, 250×10 mm) on 5 minutes 10-20% Sol B gradient. The purified product was rotate evaporated, and the yield was 47% (1.3 mg). Analytical HPLC: Purity 83% (Rt=3.8 min). MS QTOF (ESI+): Mass found: 1856.0041 [M+H]+, 1878.0041 [M+Na]+, 928.5082 [M+2H]2+, 619.3497 [M+3H]3+, 464.7638 [M+4H]4+ Calculated 1855.0473 [M+H]+, 1877.0292 [M+Na]+, 928.0273 [M+2H]2+, 619.0206 [M+3H]3+, 464.5118 [M+4H]4+.

FIGS. 18A and 18B depict the chemical structure (FIG. 18A) and characterization (FIG. 18B) of Tri-dPEG12-AMB. To synthesize Tri-dPEG12-AMB, first NH₂-dPEG12-AMB was synthesized. Fmoc-dPEG12-NHS was dissolved in DMF (1 eq., 16 μmol), TEA was added and then reacted with AMB in 0.01M PBS at pH=7.4 (3 eq., 48 μmol) for 1 hour while stirring at room temperature. The reaction crude was rotate evaporated and the Fmoc was deprotected using ~40% piperidine in DMF. The resulting NH₂-dPEG12-AMB was purified and its mass was confirmed. NH₂-dPEG12-AMB (6 eq., 16 μmol) was then reacted with TSAT (1 eq., 2.7 μmol) in DMF at room temperature for 15 mins after adding TEA. RP-HPLC: Tri-dPEG12-AMB was purified using semi-preparative Thermo Hypersil GOLD C18 column (5 um, 250×10 mm) on 10 minutes 15-30% Sol B gradient. The purified product was rotate evaporated, and the yield was 72% (1.8 mg). Analytical HPLC: Purity 80% (Rt=4.1 min). MS QTOF (ESI+): Mass found: 795.5062 [M+3H]3+, 817.4883 [M+3Na]3+, 596.8831 [M+4H]4+, 618.8647 [M+4Na]4+ Calculated 795.1239 [M+3H]3+, 817.1059 [M+3Na]3+, 596.5893 [M+4H]4+, 618.5893 [M+4Na]4+.

FIGS. 19A and 19B depict the inhibition assays for monovalent benzamidine inhibitors. FIG. 19A depicts the Ki determination with AMB utilizing a Dixon Plot. 0-1200 μM of AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 1395+165 μM. FIG. 19B depicts the Comish-Bowden S/Vo vs I plot used to determine the mode of inhibition. AMB was found to be a competitive inhibitor as the lines in this plot are parallel. All data is represented as mean±SD of triplicate experiments.

FIGS. 20A and 20B depict the inhibition assays for monovalent benzamidine inhibitors. FIG. 20A depicts the Ki determination with m-dPEG2-AMB utilizing a Dixon Plot. 0-350 μM of m-dPEG2-AMB was incubated with a fixed plasmin concentration of 42.5 nM in PBS pH 7.4. Three different S-2251 concentrations of 150 μM (orange), 250 μM (gray), and 500 μM (yellow) were utilized to obtain Ki which is the negative intersection of the lines at 259.4+35.9 μM. FIG. 20B depicts the Comish-Bowden S/Vo vs I plot used to determine the mode of inhibition. m-dPEG2-AMB was found to be a competitive inhibitor as the lines in this plot are parallel. All data is represented as mean±SD of triplicate experiments.

FIGS. 21A and 21B depict the inhibition assays for monovalent benzamidine inhibitors. FIG. 21A depicts the Ki determination with m-dPEG4-AMB utilizing a Dixon Plot. 0-350 μM of m-dPEG4-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 308.9±21.5 μM. FIG. 21B depicts the Cornish-Bowden SNo vs I plot used to determine the mode of inhibition. m-dPEG4-AMB was found to be a competitive inhibitor as the lines in this plot are parallel. All data is represented as mean±SD of triplicate experiments.

FIGS. 22A and 22B depict the inhibition assays for monovalent benzamidine inhibitors. FIG. 22A depicts the Ki determination with m-dPEG12-AMB utilizing a Dixon Plot. 0-350 μM of m-dPEG12-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 359.9±35.2 μM. FIG. 22B depicts the Cornish-Bowden SNo vs I plot to determine the mode of inhibition. m-dPEG12-AMB was found to be a competitive inhibitor as the lines in this plot are parallel. All data is represented as mean z SD of triplicate experiments.

FIGS. 23A and 23B depict the inhibition assays for monovalent benzamidine inhibitors. FIG. 23A depicts the Ki determination with m-dPEG24-AMB utilizing a Dixon Plot. 0-450 μM of m-dPEG24-AMB was incubated with a fixed plasmin concentration of 42.5 nM in PBS pH 7.4. Three different S-2251 concentrations of 100 μM (blue), 250 μM (gray), and 500 μM (yellow) were utilized to obtain Ki which is the negative intersection of the lines at 521.1±84.9 μM. FIG. 23B depicts the Cornish-Bowden SNo vs I plot used to determine the mode of inhibition. m-dPEG24-AMB was found to be a competitive inhibitor as the lines in this plot are parallel. All data is represented as mean±SD of triplicate experiments.

FIGS. 24A and 24B depict the inhibition assays for bivalent benzamidine inhibitors. FIG. 24A depicts the Ki determination with Pentamidine utilizing a Dixon Plot. 0-20 μM of Pentamidine was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 2.6±0.8 μM. FIG. 24B depicts the Comish-Bowden SNo vs I plot used to determine the mode of inhibition. Pentamidine was found to be a competitive inhibitor as the lines in this plot are parallel. All data is represented as mean±SD of triplicate experiments.

FIGS. 25A and 25B depict the inhibition assays for bivalent benzamidine inhibitors. FIG. 25A depicts the Ki determination with Bis-dPEG2-AMB utilizing a Dixon Plot. 0-75 μM of Bis-dPEG2-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 55.3±5.3 μM. FIG. 25B depicts the Cornish-Bowden SNo vs I plot used to determine the mode of inhibition. Bis-dPEG2-AMB was found to be a competitive inhibitor as the lines in this plot are parallel. All data is represented as mean±SD of triplicate experiments.

FIGS. 26A and 26B depict the inhibition assays for bivalent benzamidine inhibitors. FIG. 26A depicts the Ki determination with Bis-dPEG5-AMB utilizing a Dixon Plot. 0-75 ELM of Bis-dPEG5-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 44.3±5.3 μM. FIG. 26B depicts the Cornish-Bowden SNo vs I plot used to determine the mode of inhibition. Bis-dPEG5-AMB was found to be a competitive inhibitor as the lines in this plot are parallel. All data is represented as mean±SD of triplicate experiments.

FIGS. 27A and 27B depict the inhibition assays for bivalent benzamidine inhibitors. FIG. 27A depicts the Ki determination with Bis-dPEG13-AMB utilizing a Dixon Plot. 0-130 μM of Bis-dPEG13-AMB was incubated with a fixed plasmin concentration of 42.5 nM in PBS pH 7.4. Four different S-2251 concentrations of 100 μM (blue), 150 μM (orange), 300 μM (gray), and 500 μM (yellow) were utilized to obtain Ki which is the negative intersection of the lines at 131.4±23.8 μM. FIG. 27B depicts the Cornish-Bowden SNo vs I plot used to determine the mode of inhibition. Bis-dPEG13-AMB was found to be a competitive inhibitor as the lines in this plot are parallel. All data is represented as mean±SD of triplicate experiments.

FIGS. 28A and 28B depict the inhibition assays for bivalent benzamidine inhibitors. FIG. 28A depicts the Ki determination with Bis-dPEG25-AMB utilizing a Dixon Plot. 0-270 μM of Bis-dPEG25-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 290.4±95.8 μM. FIG. 28B depicts the Cornish-Bowden SNo vs I plot used to determine the mode of inhibition. Bis-dPEG25-AMB was found to be a competitive inhibitor as the lines in this plot are parallel. All data is represented as mean±SD of triplicate experiments.

FIGS. 29A and 29B depict the inhibition assays for trivalent benzamidine inhibitors. FIG. 29A depicts the Ki determination with Tri-AMB utilizing a Dixon Plot. 0-20 μM of Tri-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 3.9±1.7 μM. FIG. 29B depicts the Cornish-Bowden SNo vs I plot used to determine the mode of inhibition. Tri-AMB was found to be a competitive inhibitor as the lines in this plot are parallel. All data is represented as mean±SD of triplicate experiments.

FIGS. 30A and 30B depict the inhibition assays for trivalent benzamidine inhibitors. FIG. 30A depicts the Ki determination with Tri-dPEG4AMB utilizing a Dixon Plot. 0-75 μM of Tr-dPEG4-AMB was incubated with a fixed plasmin concentration of 42.5 nM in PBS pH 7.4. Four different S-2251 concentrations of 100 μM (blue), 150 μM (orange), 250 μM (gray), and 500 μM (yellow) were utilized to obtain Ki which is the negative intersection of the lines at 50.5±14 μM. FIG. 30B depicts the Comish-Bowden SNo vs I plot used to determine the mode of inhibition. Tri-dPEG4-AMB was found to be a competitive inhibitor as the lines in this plot are parallel. All data is represented as mean SD of triplicate experiments.

FIGS. 31A and 31B depict the inhibition assays for trivalent benzamidine inhibitors. FIG. 31A depicts the Ki determination with Tri-dPEG8-AMB utilizing a Dixon Plot. 0-100 μM of Tri-dPEG8-AMB was incubated with a fixed plasmin concentration of 42.5 nM in PBS pH 7.4. Three different S-2251 concentrations of 100 μM (blue), 150 μM (orange) and 500 μM (yellow) were utilized to obtain Ki which is the negative intersection of the lines at 130.8±15.9 μM. FIG. 31B depicts the Cornish-Bowden SNo vs I plot used to determine the mode of inhibition. Tri-dPEG8-AMB was found to be a competitive inhibitor as the lines in this plot are parallel. All data is represented as mean SD of triplicate experiments.

FIGS. 32A and 32B depict the inhibition assays for trivalent benzamidine inhibitors. FIG. 32A depicts the Ki determination with Tri-dPEG12-AMB utilizing a Dixon Plot. 0-210 μM of Tri-dPEG12-AMB was incubated with a fixed plasmin concentration of 42.5 nM in PBS pH 7.4. Three different S-2251 concentrations of 150 μM (orange), 250 μM (gray), and 500 μM (yellow) were utilized to obtain Ki which is the negative intersection of the lines at 241.9±34.6 μM. FIG. 32B depicts the Cornish-Bowden SNo vs I plot used to determine the mode of inhibition. Tri-dPEG12-AMB was found to be a competitive inhibitor as the lines in this plot are parallel. All data is represented as mean SD of triplicate experiments.

FIGS. 33A-33E depict monovalent TXA and multivalent TXA inhibitors of valencies 2, 4, 8 and 16.

FIGS. 34A and 34B depict the chemical structure (FIG. 34A) of AMB-PEG4-TXA and (FIG. 34B) of AMB-PEG8-TXA as exemplary examples of hetero-multivalent molecules.

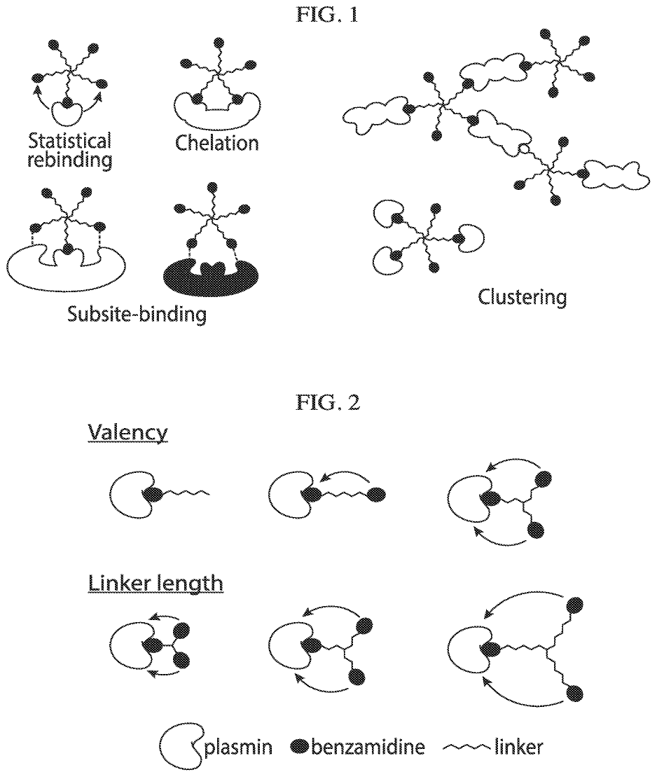

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, compositions and methods have been discovered that surprisingly allow for modulating target molecules. The compositions are useful for the treatment of bleeding disorders or thrombosis associated disorders. Advantageously, the compositions exhibit multivalent effect and produces enzyme binders with stronger and/or selective inhibition of target enzymes.

Multivalent affinity molecules of the present disclosure can be designed the modulate activities of a target molecule by chemically coupling the affinity moieties to the scaffold to achieve multivalency. As used herein, the term “target molecule” refers to a molecule to which a multivalent affinity molecule of the present application is directed to and/or binds, resulting in a change in the behavior or function of the target molecule. Particularly suitable target molecules having activities that can be modulated using the multivalent affinity molecules of the present disclosure are enzymes. Particularly suitable enzymes to which a multivalent affinity molecule of the present application is directed to and/or binds include proteases. Exemplary embodiments of multivalent affinity molecules of the present disclosure are demonstrated herein using affinity moieties that bind plasmin, thrombin, and tissue-type plasminogen activator (tPA). These affinity moieties can be divided into two categories: (1) those that bind lysine binding sites (LBS) and (2) those that bind the active site. Affinity moieties can bind to the active site of an enzyme. Other affinity moieties can bind to alternative sites of a target molecule. Exemplary affinity moieties include protease inhibitors such as serine protease inhibitors and binders of known protein domains.

In one aspect, the present disclosure is directed to a multivalent affinity molecule having from two affinity moieties to about 100 affinity moieties; and a scaffold, wherein the two affinity moieties to about 100 affinity moieties are covalently coupled to the scaffold. Suitably, the multivalent affinity molecule can have from about two affinity moieties to about 36 affinity moieties, including from about two affinity moieties to about 10 affinity moieties.

In one embodiment, the affinity moieties of the multivalent affinity molecules of the present disclosure are all the same affinity moiety (referred to and described herein as “homo-multivalent affinity molecules”). For example, where the multivalent affinity molecule includes three affinity moieties, in this exemplary embodiment, all three of the affinity moieties are the same affinity moiety. Another exemplary embodiment is a multivalent affinity molecule having 100 affinity moieties wherein all 100 are the same affinity moiety. In another embodiment, the affinity moieties of the multivalent affinity molecule are different affinity moieties (referred to and described herein as “hetero-multivalent affinity molecules”). For example, where the multivalent affinity molecule includes three affinity moieties, in this exemplary embodiment, two of the three of the affinity moieties can be the same affinity moiety and one of the three affinity moieties being a different affinity moiety from the other two affinity moieties. In another example, all three affinity moieties can be different affinity moieties. Another exemplary embodiment is a multivalent affinity molecule having 100 affinity moieties wherein at least one affinity moiety of the 100 affinity moieties is different from at least one other affinity moiety.

Suitable affinity moieties include small molecule compounds, proteins, peptides, and combinations thereof, that specifically interact with a target molecule. In particular, the affinity moiety is designed to specifically interact with a binding site of the target molecule. As used herein, “binding site” refers to sites in a target molecule where an affinity moiety and/or a portion of an affinity moiety can non-covalently and reversibly couple. As used herein, “reversibly coupled” refers to the capability of the affinity moiety of the multivalent affinity molecule to bind and unbind the target molecule. For example, the target molecule and the affinity moiety can be reversibly coupled by electrostatic attraction, hydrogen bonding, hydrophobic effects and Van der Waals forces. For example, in a multivalent affinity molecule using benzamidine as an affinity moiety, the benzamidine affinity moiety can bind plasmin via its active site and also have the capability to be released when the plasmin kringle moieties contact lysine residues on fibrin. Thus, in the absence of fibrin, plasmin has the propensity to remain bound to benzamidine. The binding site can bind such that the activity of the target molecule is modulated, increased, enhanced, inhibited, interfered with and/or reduced while the target molecule is bound to the affinity moiety of the multivalent affinity molecule. The binding site can also be another site to which the affinity moiety interacts without impacting the activity of the target molecule. The binding site of the target molecule can be an active site of the target molecule, a cofactor binding site of the target molecule, a coenzyme binding site of the target molecule, a substrate binding site of the target molecule, an autoinhibitory site of the target molecule, a regulatory site of the target molecule, and any other binding domain on the target molecule. Specific binding of the affinity moiety to the binding site of the target molecule can modulate, increase, enhance, inhibit, interfere with and/or reduce the target molecule's activity in a competitive inhibitory manner, an uncompetitive inhibitory manner, a non-competitive inhibitory manner or a partially competitive manner. Specific binding of the affinity moiety to the binding site of the target molecule advantageously sequesters the target molecule to inhibit its activity while bound to the multivalent affinity molecule. Specific binding of the affinity moiety to the binding site of the target molecule can also advantageously orient other domains of the target molecule to be accessible for interacting with the target molecule's substrate. For target molecules having autolysis activities, specific binding of the affinity moiety to the catalytic site of the target molecule can modulate, inhibit, interfere with and/or reduce autolysis activities that lead to self-inactivation and clearance of the target molecule allowing for improved half-life of the target molecule.

Suitable small molecule compound affinity moieties can have a molecular weight of from about 50 Daltons to about 5,000 Daltons. Suitable small molecule affinity moieties can contain at least one aryl ring with an attached group containing at least two nitrogen atoms, wherein the nitrogen groups form a triangular structure such as an amidine and wherein one nitrogen atom has a double bond with a carbon atom that is attached to an aryl ring. They can also be reactive cyclohexanones, nitrile warheads, aldehyde peptidomimetics, cyclic peptidomimetics, polypeptides of the Kunitz and Kazal-type and lysine analogs.

Suitable affinity moieties include and/or are selected from the group consisting of a cyclohexanone, a cyclohexane, a quinidine, an amidine, a peptide, a tripeptide that comprises a nitrile warhead, a cyclic peptidomimetic, and combinations thereof. Particularly suitable plasmin affinity moieties include benzamidine, benzylamine, tranexamic acid (TXA), ε-aminocaproic acid (EACA), lysine, and combinations thereof. A particularly suitable benzamidine is 4-aminomethyl benzamidine. Benzamidine and its derivatives are common reversible, competitive inhibitors of trypsin family proteases that bind to the active site of plasmin via an amidine group. Benzamidine has also been shown to exhibit weak subsite binding to the light chain and kringle 5 of plasmin. TXA is a clinically approved lysine analogue used to treat hyper-fibrinolysis associated bleeding.

Suitably, affinity moieties can have a planar separation length or hydrodynamic diameter ranging from about 1 nanometer in length to about 1,000 nanometers in length when measured from one affinity moiety to another affinity moiety. Planar separation length is determined using bond length of the scaffold molecule. Each affinity moiety is covalently coupled to a linker molecule to form a scaffold shared among the affinity moieties of the multivalent affinity molecule.

Suitable affinity moieties can be, for example, serine protease inhibitors. Particularly suitable affinity moieties can be, for example, serine protease inhibitors that specifically bind to the active site of the serine protease. Particularly suitable affinity moieties can be, for example, benzamidines (see, Table 1). Suitable benzamidines can be, for example, benzamidine, 4-aminobenzamidine, 4-carboxybenzamidine, 4-aminomethyl benzamidine, and combinations thereof. Other suitable small molecule compound inhibitors can be, for example, bivalirudin, argatroban ((2R,4R)-1-[(25)-5-(diaminomethylideneamino)-2-[[(3R)-3-methyl-1,2,3,4-tetrahydroquinolin-8-yl]sulfonylamino]pentanoyl]-4-methyl-piperidine-2-carboxylic acid), melagatran (or its prodrug ximelagatran; ethyl 2-[[(1R)-1-cyclohexyl-2-[(2S)-2-[[4-(N′-hydroxycarbamimidoyl)phenyl]methylcarbamoyl]azetidin-1-yl]-2-oxo-ethyl]amino]acetate), dabigatran (Ethyl 3-{[(2-f[(4-{N′-hexyloxycarbonyl carbamimidoyl}phenyl)amino]methyl}-1-methyl-1H-benzimidazol-5-yl)carbonyl](pyridin-2-yl-amino)propanoate), and combinations thereof. Other suitable small molecule compound affinity moieties can be, for example, amprenavir ((3S)-oxolan-3-yl N-[(2S,3R)-3-hydroxy-4-[N-(2-methylpropyl)(4-aminobenzene)sulfonamido]-1-phenylbutan-2-yl]carbamate), atazanavir (methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate), darunavir ([(1R,5S,6R)-2,8-dioxabicyclo[3.3.0]oct-6-yl]N-[(2S,3R)-4-[(4-aminophenyl)sulfonyl-(2-methylpropyl)amino]-3-hydroxy-1-phenyl-butan-2-yl]carbamate), fosamprenavir ({[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid), indinavir ((2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide), lopinavir/ritonavir combination ((2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide and 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate), nelfinavir ((3S,4aS,8aS)-N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide), ritonavir (1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate), saquinavir ((2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide), tipranavir (N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide), and combinations thereof. Other new small molecule inhibitors that are useful as affinity moieties are continually being developed as protease inhibitors to combat numerous diseases and illnesses, and can be used as described in the present disclosure.

In another particularly suitable embodiment, the affinity moiety is a protein, a peptide or a peptidomimetic that specifically binds a target molecule. Particularly suitable proteins and peptides can be for example antibody molecules that specifically bind to the target molecule. Antibody molecules include, for example, polyclonal antibodies and monoclonal antibodies. Antibody molecules also include chimeric antibodies, humanized antibodies, antigen binding fragments (Fab), antibody variable domains (Fv), single chain variable fragments (scFv), and complement determining regions (CDRs). Other suitable peptide inhibitors can be, for example, serpins, hirudin, bivalirudin, lepirudin, desirudin, and combinations thereof.

The affinity moieties are covalently coupled to the scaffold to form the multivalent affinity molecule of the present disclosure. The term, “covalently coupled to” is used according to its ordinary meaning as understood by those skilled in the art to refer to the coupling of, connecting of, attaching of, and joining of the affinity moiety to the scaffold (linker) molecule whereby a chemical bond forms between the scaffold (linker) molecule and the affinity moiety. The scaffold (linker) molecule and the affinity moiety are chemically reacted to form a chemical bond (covalently linked) between the scaffold (linker) molecule and the affinity moiety.

In one embodiment, the multivalent affinity molecule of the present disclosure is designed to modulate the activity of an enzyme involved in blood coagulation and fibrinolysis. As used herein, “modulate” refers to varying the activity of an enzyme. In one exemplary embodiment, the multivalent affinity molecules of the present disclosure can inhibit the activity of an enzyme. In another exemplary embodiment, the multivalent affinity molecules of the present disclosure can reduce the activity of an enzyme. In yet another exemplary embodiment, the multivalent affinity molecules of the present disclosure can increase the activity of an enzyme. Suitable enzymes that can be modulated using the multivalent affinity molecule of the present disclosure include proteases. Particularly suitable proteases the multivalent affinity molecules of the present disclosure can modulate are natural and synthetic proteases that contain a catalytic triad domain. As known to those skilled in the art, a catalytic triad refers to the three amino acid residues that function together at the center of the active site in enzymes such as, proteases, amidases, esterases, acylases, lipases and β-lactamases. Suitable proteases the multivalent affinity molecules of the present disclosure can modulate include serine proteases, for example. As known by those skilled in the art, serine proteases are enzymes that cleave peptide bonds in proteins, in which a serine amino acid residue serves as the nucleophilic amino acid at the enzyme's active site. Particularly suitable serine proteases the multivalent affinity molecules of the present disclosure can modulate include, for example, plasmin, urokinase, tissue plasminogen activator (plasminogen activator), trypsin, a trypsin like enzyme, batroboxin (reptilase) and combinations thereof. A particularly suitable serine protease the multivalent affinity molecules of the present disclosure can modulate includes plasmin such as, for example, plasmin, delta plasmin, mini-plasmin, and microplasmin. Through recombinant manipulation, the plasmin variant, delta-plasmin (6-plasmin), mini-plasmin, and microplasmin, have been produced in which K2-K5 have been deleted from full-length plasmin, while retaining the moderate-affinity of K1 to bind fibrin. Elimination of K2-K5 enables the technical feasibility to synthesize, purify and refold active enzyme from an E. coli expression vector.

Suitable serine proteases the multivalent affinity molecules of the present disclosure can modulate include those that participate in the coagulation/fibrinolysis system. Particularly suitable serine proteases the multivalent affinity molecules of the present disclosure can modulate include, for example, plasmin, polyphosphate, kallekreins (including plasma kallekrein (KLKB1) and tissue kallekreins (KLK1, KLK2, KLK3, KLK4, KLK5, KLK6, KLK7, KLK8, KLK9, KLK10, KLK11, KLK12, KLK13, KLK14, and KLK15)), nucleic acids, tissue factor, factor XI, factor XIa, factor XII, factor XIIa, factor Xa, factor IX, urokinase, prothrombin, thrombin, a thrombin like enzyme, batroboxin (reptilase), tissue plasminogen activator, protein C, protein S, protein Z, trypsin, chymotrypsin, elastase, peptidase, subtilisin, trypsin, tryptase, and combinations thereof.

A suitable affinity moiety includes benzamidines. Benzamidines are serine protease inhibitors that can specifically bind the active site of plasmin. Binding affinity of the multivalent benzamidine affinity ligands can be tightly controlled by modifying multivalent properties such as valency and scaffold (linker) length of benzamidine. For the synthesis of multivalent affinity molecules using a benzamidine as an affinity moiety, the benzamidine derivative molecules as depicted in Table 1 can be utilized. Similarly, multivalent affinity molecules using tranexamic acid inhibitors can also be synthesized and used to tightly control plasmin inhibition by modifying valency and scaffold (linker) length of multivalent TXA molecules. Further, a combination of benzamidine and TXA can be used in the multivalent inhibitors to achieve desired binding/inhibition.

TABLE 1
Commercially available affinity moieties

| Affinity Moieties | Structure |
|---|---|
| Benzamidine | 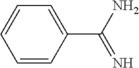 |
| 4-Aminobenzamidine | 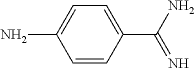 |
| 4-Carboxybenzamidine | 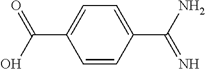 [SMILES: N=C(N)c1ccc(C(=O)O)cc1] |
| 4-Aminomethyl Benzamidine | 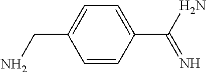 [SMILES: N=C(N)c1ccc(CN)cc1] |
| Tranexamic Acid (TXA) | 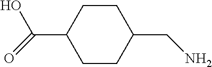 [SMILES: NCC1CCC(C(=O)O)CC1] |
| ϵ-Aminocaproic Acid | 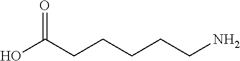 |
| Pentamidine (Nebupent) | 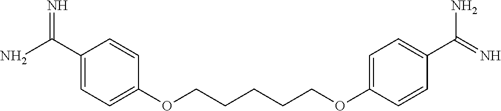 [SMILES: N=C(N)c1ccc(OCCCCCOc2ccc(C(=N)N)cc2)cc1] |

The multivalent affinity molecules of the present disclosure also include a scaffold molecule. Suitable scaffold (linker) molecules include polymer linkers. Particularly suitable polymer linkers include polyethylene glycol (PEG) and hydrolysable linkers. Linkers can be rigid or flexible.

Suitable scaffold molecules also include dendrimers. Suitable dendrimers include polyamidoamine (PAMAM) dendrimers. Other suitable dendrimer scaffolds include a polypropylamine (POPAM), PAMAM-POPAM, poly(propylene imine) (PPI), polvether-copolyester (PEPE), PEGylated, peptide, triazine, citric acid, polyester, polyether, phosphorous glycodendrimer, liquid crystalline, carbosilane, fulleropyrrolidine arborol, bis-MPA dendrimers, polyetherimine (PETIM), and combinations thereof.

Suitable scaffold molecules also include lipid molecules. The lipid scaffold can include a mixture of lipid species. Suitable lipid molecules include phosphatidylcholine, phosphatidic acid, phosphatidylethanolamine, phosphatidylserine, a phosphoinositide, a phosphingolipid and combinations thereof. Suitable phospholipids are known by those skilled in the art and are commercially available (AVANTI® Polar Lipids, Inc., Alabaster, Ala.). Phospholipids can be, for example, phosphatidylcholine, phosphatidic acid, phosphatidylethanolamine, phosphatidylserine, phosphoinositides including phosphatidylinositol, phosphatidylinositol phosphate, phosphatidylinositol bisphosphate, and phosphatidylinositol triphosphate, and phosphingolipids including ceramide phosphorylcholine, ceramide phosphorylethanolamine, and ceramide phosphoryl lipid. Suitable phospholipids can also include synthetic lipids such as, for example, palmitic acid, organic/inorganic nanoparticles, and co-block polymer-based nanoparticles such as, for example, polylactic acid) (PLA), poly(glycolic acid) (PGA), poly(lactide-co-glycolide) (PLGA), polyethylene glycol) (PEG), dextran, poly(ε-caprolactone) (PCL), poly (β-benzyl L-aspartate) (PLBA), poly (γ-benzyl L-glutamate) (PLBG), poly (alkylcyanoacrylate), poly esters, poly (ortho-esters) (POE), polyanhydrides (PA), polyamides, and silica. Particularly suitable phospholipids can be phospholipid derivatives such as, for example, natural phospholipid derivatives and synthetic phospholipid derivatives. Phospholipid derivatives can be, for example, 1,2-Didecanoyl-sn-glycero-3-phosphocholine (DDPC), 1,2-Dierucoyl-sn-glycero-3-phosphate (Sodium Salt) (DEPA-NA) 1,2-Dierucoyl-sn-glycero-3-phosphocholine (DSPC); 1,2-Dierucoyl-sn-glycero-3-phosphoethanolamine (DEPE); 1,2-Dierucoyl-sn-glycero-3 [Phospho-rac-(1-glycerol) (Sodium Salt) (DEPG-NA); 1,2-Dilinoleoyl-sn-glycero-3-phosphocholine (DEPC); 1,2-Dimyristoyl-sn-glycero-3-phosphate (Sodium Salt) (DEPA-NA); 1,2-Dilauroyl-sn-glycero-3-phosphocholine (DLPC); 1,2-Dilauroyl-sn-glycero-3-phosphoethanolamine (DLPE); 1,2-Dilauroyl-sn-glycero-3[Phospho-rac-(1-glycerol) (Sodium Salt) (DLPG-NA); 1,2-Dilauroyl-sn-glycero-3[Phospho-rac-(1-glycerol) (Ammonium Salt) (DLPG-NH4); 1,2-Dilauroyl-sn-glycero-3-phosphoserine (Sodium Salt) (DLPS-NA); 1,2-Dimyristoyl-sp-glycero-3-phosphate (Sodium Salt) (DMPA-NA); 1,2-Dimyristoyl-sn-glycero-3-phosphocholine (DMPC); 1,2-Dimyristoyl-sn-glycero-3-phosphoethanolamine (DAVE); 1,2-Dimyristoyl-sn-glycero-3[Phospho-rac-(1-glycerol) (Sodium Salt) (DMPG-NA); 1,2-Dimyristoyl-sn-glycero-3[Phospho-rac-(1-glycerol) (Ammonium Salt) (DMPG-NH4); 1,2-Dimyristoyl-sn-glycero-3[Phospho-rac-(1-glycerol) (Sodium/Ammonium Salt) (DMPG-NH4/NA); 1,2-Dimyristoyl-sn-glycero-3-phosphoserine (Sodium Salt) (DMPS-NA); 1,2-Dioleoyl-sn-glycero-3-phosphate (Sodium Salt) (DOPA-NA); 1,2-Dioleoyl-sn-glycero-3-phosphocholine (DOPC); 1,2-Dioleoyl-sn-glycero-3-phosphoethanolamine (DOPE); 1,2-Dioleoyl-sn-glycero-3[Phospho-rac-(1-glycerol) (Sodium Salt) (DOPG-NA); 1,2-Dioleoyl-sn-glycero-3-phosphoserine (Sodium Salt) (DOPS-NA); 1,2-Dipalmitoyl-sn-glycero-3-phosphate (Sodium Salt) (DPPA-NA); 1,2-Dipalmitoyl-sn-glycero-3-phosphocholine (DPPC); 1,2-Dipalmitoyl-sn-glycero-3-phosphoethanolamine (DPPE); 1,2-dipalmitoyl-sn-glycero-3-phosphoethanolamine-N-(glutaryl) (sodium salt) (DPPG-GA) 1,2-Dipalmitoyl-sn-glycero-3[Phospho-rac-(1-glycerol) (Sodium Salt) (DPPG-NA); 1,2-Distearoyl-sn-glycero-3-phosphate (Sodium Salt) (DPPG-NH4); 1,2-Dipalmitoyl-sn-glycero-3-phosphoserine (Sodium Salt) (DPPS-NA); 1,2-Distearoyl-sn-glycero-3-phosphate (Sodium Salt) (DSPA-NA); 1,2-Distearoyl-sn-glycero-3-phosphocholine (DSPC); 1,2-Distearoyl-sn-glycero-3-phosphoethanolamine (DSPE); 1,2-Distearoyl-sn-glycero-3[Phospho-rac-(1-glycerol) (Sodium Salt) (DSPC-NA); 1,2-Distearoyl-sn-glycero-3[Phospho-rac-(1-glycerol) (Ammonium Salt) (DSPG-NH4); 1,2-Distearoyl-sn-glycero-3-phosphoserine (Sodium Salt) (DSPS-NA); Egg-PC (EPC); Hydrogenated Egg PC (HEPC); Hydrogenated Soy PC (HSPC); 1-Myristoyl-sn-glycero-3-phosphocholine (LYSOPC MYRISTIC); 1-Palmitoyl-sn-glycero-3-phosphocholine (LYSOPC PALMITIC); 1-Stearoyl-sn-glycero-3 phosphocholine (LYSOPC STEARIC); 1-Myristoyl-2-palmitoyl-sn-glycero 3-phosphocholine (Milk Sphingomyelin MPPC); 1-Myristoyl-2-stearoyl-sn-glycero-3-phosphocholine (MSPC); 1-Palmitoyl-2-myristoyl-sn-glycero-3-phosphocholine (PMPC); 1-Palmitoyl-2-oleoyl-sn-glycero-3-phosphocholine (POPC); 1-Palmitoyl-2-oleoyl-sn-glycero-3-phosphoethanolamine (POPE); 1-Palmitoyl-2-oleoyl-sn-glycero-3 [Phospho-rac-(1-glycerol)](Sodium Salt) (POPG-NA); 1-Palmitoyl-2-stearoyl-sn-glycero-3-phosphocholine (PSPC); 1-Stearoyl-2-myristoyl-sn-glycero-3-phosphocholine (SMPC) 1-Stearoyl-2-oleoyl glycerol-3-phosphocholine (SOPC); 1-Stearoyl-2-palmitoyl-sn-glycero-3-phosphocholine (SPPC); and combinations thereof. Particularly suitable phospholipids can also be modified with a water soluble polymer such as poly(ethylene glycol) to form PEGylated phospholipids such as, for example, DSPE-PEG(2000); DSPE-PEG(2000)-amine, DSPE-PEG(2000) carboxy NHS and combinations thereof. Phospholipids can also be modified with polymeric sugars such as poly(lactic-co-glycolic acid) (PLEA)

In some embodiments, multivalent affinity molecules include a bulk lipid. The term “bulk lipid” is used according to its ordinary meaning as would be understood by one of ordinary skill in the art to mean the lipids that form the main structure of a membrane without contacting membrane proteins. The amount of bulk lipid can range from 0 to about 10 M. In some embodiments, the bulk lipid can be PEGylated. The density of bulk lipid can be about 100 molecules per square nanometer on the surface of the multivalent affinity molecule to about 1 molecule per 20000 square nanometer on the surface of the multivalent affinity molecule.

The multivalent affinity molecule can be a micelle, a liposome, inorganic nanoparticles such as metallic, magnetic, quantum dot, crystalline nanoparticle, polymeric nanoparticle (both biodegradable and non-biodegradable), and combinations thereof.

The multivalent affinity molecules include homo-multivalent affinity molecules and hetero-multivalent affinity molecules. As used herein, “homo-multivalent” affinity molecules include multiple affinity moieties that are the same affinity molecule. For example, a homo-multivalent affinity molecule would have as the affinity moiety only an affinity moiety that binds to the active site of an enzyme. An exemplary homo-multivalent affinity molecule would include only benzamidine affinity moieties coupled to the same scaffold. As used herein, “hetero-multivalent” affinity molecules include multiple affinity moieties that are different affinity molecules. For example, a hetero-multivalent affinity molecule would have as the affinity moieties at least one affinity moiety that binds to the active site of an enzyme and at least one additional affinity moiety that binds to a site that is not the active site (such as a kringle domain of plasmin). An exemplary hetero-multivalent affinity molecule would include a benzamidine affinity moiety and a TXA affinity moiety covalently coupled to the same scaffold.

It should be understood that the multivalent affinity molecules can have stronger and/or selective inhibition of enzymes, for example, via statistical binding. In other aspects, it can be via subsite binding, chelation or clustering effects. It should be further understood that one multivalent affinity molecule can bind one, two, or more target molecules (e.g., enzymes). For example, one affinity moiety of the multivalent affinity molecule can bind the active site of one target molecule and a second affinity moiety of the multivalent affinity molecule can bind the active site of a second (different) target molecule.

In another aspect, the present disclosure is directed to a method of treating a hyper-fibrinolysis associated bleeding disorder in a subject in need thereof. The method includes administering to the subject in need thereof a multivalent affinity molecule comprising: two affinity moieties to about 100 affinity moieties selected from benzamidine, benzylamine, tranexamic acid, lysine, ε-aminocaproic acid, and combinations thereof; and a scaffold, wherein the two affinity moieties to about 100 affinity moieties are covalently coupled to the scaffold; and a scaffold, wherein the two affinity moieties to about 100 affinity moieties are covalently coupled to the scaffold; and a scaffold, wherein the two affinity moieties to about 100 affinity moieties are covalently coupled to the scaffold.

The subject in need thereof has or is suspected of having a hyper-fibrinolysis associated bleeding disorder. The subject in need thereof can be treated with the multivalent affinity molecules of the present disclosure for hyperfibrinolysis-associated bleeding during surgery, disseminated intravascular coagulation, hemophilia, menorrhagia, and combinations thereof.

The multivalent affinity molecules of the present disclosure are administered to a subject in need. As used herein with regard to treating a hyper-fibrinolysis associated bleeding disorder, “a subject in need thereof” refers to a subset of individuals in need of treatment/protection for a hyper-fibrinolysis associated bleeding disorder. Some individuals that are in specific need of treatment may include subjects who are susceptible to, or at elevated risk of hyperfibrinolysis-associated bleeding during surgery, trauma, disseminated intravascular coagulation, hemophilia, menorrhagia, and combinations thereof. Individuals can be susceptible to, or at elevated risk of, experiencing symptoms due to family history, age, environment, and/or lifestyle. Based on the foregoing, because some of the methods embodiments of the present disclosure are directed to specific subsets or subclasses of identified individuals (that is, the subset or subclass of subjects “in need thereof” of assistance in addressing one or more specific conditions noted herein), not all individuals will fall within the subset or subclass of individuals as described herein for certain diseases, disorders or conditions.

In another aspect, the present disclosure is directed to a method of treating a thrombosis associated disorder in a subject in need thereof. The method includes administering to the subject in need thereof a multivalent affinity molecule comprising: two affinity moieties to about 100 affinity moieties that modulate enzymes that promote coagulation and combinations thereof; and a scaffold, wherein the two affinity moieties to about 100 affinity moieties are covalently coupled to the scaffold. It should be understood that the same affinity molecule types listed previously are suitable for treating a thrombosis associated disorder, as they are serine proteases that are both clot forming and clot digesting so the molecular structure of the affinity ligands does not necessarily change but the valency and mixture of inhibitors or differences in linker length can contribute to selective inhibition of thrombin over plasmin, for example, if administering a naked affinity molecule to modulate a target enzyme in vivo. Alternatively, the same affinity ligands can be loaded with different enzymes to administer thrombin affinity complex (treat bleeding disorder) or plasmin affinity complex (treat thrombosis disorder).

As used herein with regard to treating a thrombosis associated disorder, “a subject in need thereof” refers to a subset of individuals in need of treatment/protection for a thrombosis associated disorder. Some individuals that are in specific need of treatment may include subjects who are susceptible to, or at elevated risk of heart attack, stroke, deep vein thrombosis, pulmonary embolism, indwelling catheter occlusion, and combinations thereof. Individuals can be susceptible to, or at elevated risk of, experiencing symptoms due to family history, age, environment, and/or lifestyle. Based on the foregoing, because some of the methods embodiments of the present disclosure are directed to specific subsets or subclasses of identified individuals (that is, the subset or subclass of subjects “in need thereof” of assistance in addressing one or more specific conditions noted herein), not all individuals will fall within the subset or subclass of individuals as described herein for certain diseases, disorders or conditions.

The term “administering” as used herein includes all means of introducing the multivalent affinity molecule described herein to the subject. A particularly suitable administration routes include intravenous (IV), intramuscular (IM), intraperitoneal (IP), topical, and oral administration. The multivalent affinity molecule described herein may be administered in unit dosage forms and/or formulations containing conventional nontoxic pharmaceutically-acceptable carriers, adjuvants, excipients, vehicles, and combinations thereof.

In one aspect, the present disclosure is directed to modulating enzyme activity. The method includes contacting the enzyme with a multivalent affinity molecule comprising: two affinity moieties to about 100 affinity moieties that specifically bind the enzyme; and a scaffold, wherein the two affinity moieties to about 100 affinity moieties are covalently coupled to the scaffold and wherein upon binding of at least one of the two affinity moieties to about 100 affinity ligands to the enzyme reduces the activity of the enzyme.

In one embodiment, the method is an in vitro method of modulating enzyme activity. In another embodiment, the method is an in vivo method of modulating enzyme activity.

In one aspect, the present disclosure is directed to a method of sequestering an enzyme. The method includes: contacting the enzyme with a multivalent affinity molecule, the multivalent affinity molecule comprising: two affinity moieties to about 100 affinity moieties that specifically bind the enzyme; and a scaffold, wherein the two affinity moieties to about 100 affinity moieties are covalently coupled to the scaffold and wherein at least one of the two affinity moieties to about 100 affinity moieties are reversibly coupled to the enzyme to sequester the enzyme.

In one aspect, the present disclosure is directed to a method of delivering an enzyme. The method includes: contacting the enzyme with a multivalent affinity molecule, the multivalent affinity molecule comprising: two affinity moieties to about 100 affinity moieties that reversibly bind the enzyme; and a scaffold, wherein the two affinity moieties to about 100 affinity moieties are covalently coupled to the scaffold and wherein the two affinity moieties to about 100 affinity moieties are reversibly coupled to the enzyme to form an enzyme-multivalent affinity molecule complex; providing the enzyme-multivalent affinity molecule complex to a target site; wherein the enzyme is released from the enzyme-multivalent affinity molecule complex at the target site to deliver the enzyme.

In one aspect, the present disclosure is directed to a method of prophylaxis of a bleeding disorder in a subject in need thereof, the method comprising: administering to the subject in need thereof a multivalent affinity molecule comprising two affinity moieties to about 100 affinity moieties covalently coupled to a scaffold. A subject in need thereof can be susceptible to, or at elevated risk of, experiencing a bleeding disorder due to family history, age, environment, and/or lifestyle. Based on the foregoing, because some of the methods embodiments of the present disclosure are directed to specific subsets or subclasses of identified individuals (that is, the subset or subclass of subjects “in need thereof” of assistance in addressing one or more specific conditions noted herein), not all individuals will fall within the subset or subclass of individuals as described herein for certain diseases, disorders or conditions. A subject in need of prophylaxis of a bleeding disorder include subjects who are susceptible to, or at elevated risk of surgery, disseminated intravascular coagulation, hemophilia, menorrhagia, and combinations thereof.

In one aspect, the present disclosure is directed to a method of prophylaxis of thrombosis in a subject in need thereof, the method comprising: administering to the subject in need thereof a multivalent affinity molecule comprising two affinity moieties to about 100 affinity moieties covalently coupled to a scaffold. A subject in need thereof can be susceptible to, or at elevated risk of, experiencing thrombosis due to family history, age, environment, and/or lifestyle. Based on the foregoing, because some of the methods embodiments of the present disclosure are directed to specific subsets or subclasses of identified individuals (that is, the subset or subclass of subjects “in need thereof” of assistance in addressing one or more specific conditions noted herein), not all individuals will fall within the subset or subclass of individuals as described herein for certain diseases, disorders or conditions. A subject in need of prophylaxis of thrombosis include subjects who are susceptible to, or at elevated risk of heart attack, stroke, deep vein thrombosis, pulmonary embolism, indwelling catheter occlusion, and combinations thereof.

In general, “in need of prophylaxis” refers to the judgment made by a caregiver (e.g., physician, nurse, nurse practitioner, etc. in the case of humans; veterinarian in the case of animals, including non-human mammals) that the subject will become ill and/or has high/reasonable risk of a future bleeding or thrombotic event that would benefit from a therapeutic intervention. This judgment is made based on a variety of factors based on the caregiver's expertise and knowledge that the subject is ill, or will be ill, as the result of a disease, condition or disorder as described herein. In this context, the multivalent affinity molecule of the present disclosure is used in a protective or preventive manner.

The disclosure will be more fully understood upon consideration of the following non-limiting Examples.

### EXAMPLES

### Example 1

Plasmin is an endogenous enzyme responsible for digesting fibrin present in blood clots. Monovalent plasmin inhibitors are utilized clinically to treat hyperfibrinolysis-associated bleeding events. Benzamidine is a reversible inhibitor that binds to plasmin's active site. This Example demonstrates that multivalency can be used to enhance plasmin inhibition using multiple affinity moieties linked on a common scaffold. Plasmin plays a vital role in hemostasis, immune, and inflammatory responses. It is the key enzyme in the fibrinolytic pathway responsible for degrading fibrin in blood clots. Plasmin belongs to the serine protease family to which over one third of known proteases belong. Serine proteases have a nucleophilic Serine (Ser) residue at the enzyme active site and typically utilize the Asp-His-Ser catalytic triad to hydrolyze the peptide bond of the substrate. Serine protease family encompasses a wide array of enzymes that are involved in fibrinolysis, blood coagulation, digestion, development, fertilization, apoptosis, and immunity. Trypsin, thrombin, tissue plasminogen activator are a few other examples of serine proteases. Plasmin is activated from its plasminogen precursor and possesses a light chain (~25 kDa) which contains the serine protease active site and a heavy chain (~60 kDa) comprising 5 kringle domains. Active site plasmin inhibitors are useful for treating hyperfibrinolysis-associated bleeding disorders as well as cancer and inflammatory disorders caused by excessive plasmin activity.

Multivalency (multivalent avidity) is defined as the enhanced response observed with multiple affinity ligands linked on a common scaffold compared to the total response observed with equivalent number of monovalent affinity ligands. Leveraging this phenomenon eliminates the need for producing large amounts of monovalent ligands or ligands with very high affinity. Multivalency can also enhance overall binding.

Benzamidine and its derivatives are common reversible, competitive inhibitors of trypsin family proteases that bind to the active site of plasmin via an amidine group. Alves et al. have shown that pentamidine, an FDA approved bivalent benzamidine, was a 15-fold stronger plasmin inhibitor than the strongest monovalent benzamidine owing to multivalent avidity effects, presumably statistical rebinding as pentamidine is a short inhibitor (0.9 nm).

Herein, multivalent benzamidines of varying valency (mono-, bi-, tri-, and larger) and scaffold linker lengths (1-12 nm) were synthesized to study their effect on plasmin inhibition. For this, polyethylene glycol (PEG) scaffolds were used to ensure uniform distribution and to have precise control on the length of linker molecules forming the scaffold shared between affinity moieties to isolate its effects on inhibition. As linker length dictates the mechanism of multivalent inhibition, multivalent benzamidines of lower valency that have short and flexible PEG linkers were specifically synthesized to promote statistical rebinding and minimize clustering and other modes of inhibition via binding by the multivalent affinity molecules. Although, benzamidine has also been shown to exhibit weak subsite binding to the light chain and kringle 5 of plasmin, prior comparison of benzamidine inhibition across both plasmin and delta-plasmin, a recombinant plasmin variant possessing only the kringle 1 domain and active site, demonstrates that the active site is the primary benzamidine binding mode and therefore, subsite binding impact has minimal effect with multivalent benzamidine molecules.

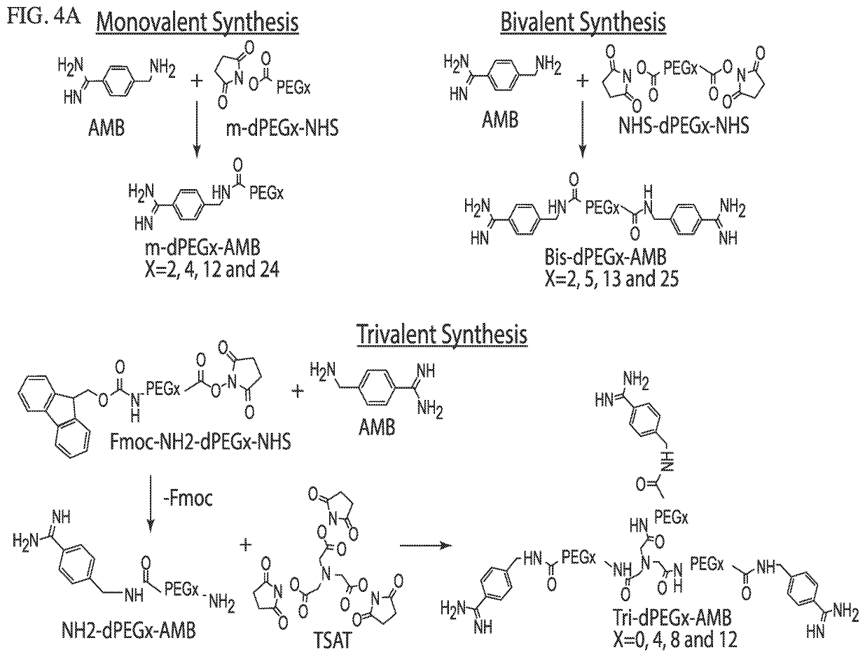

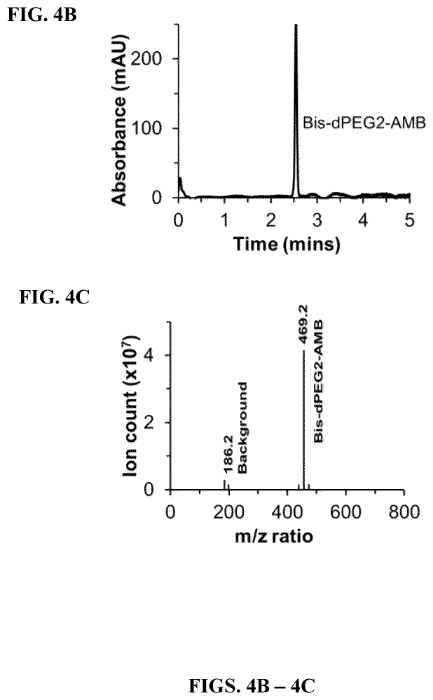

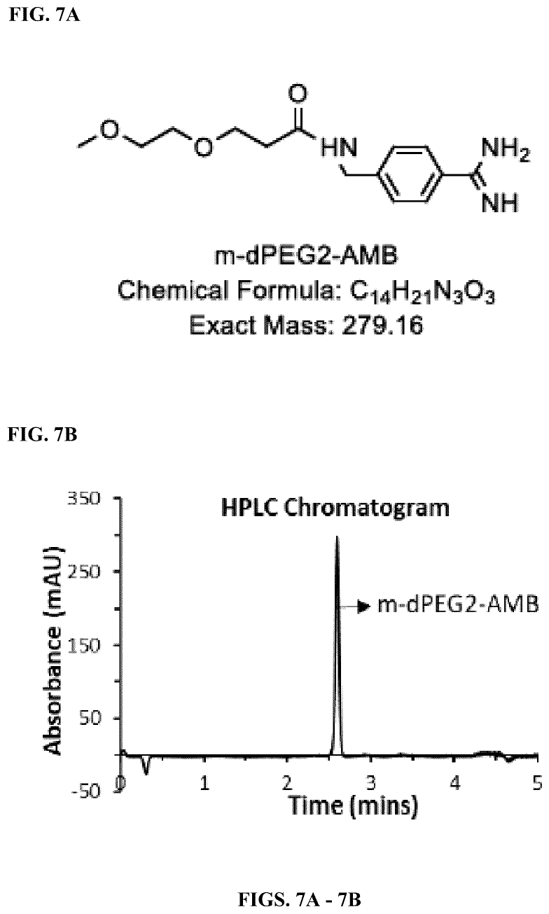

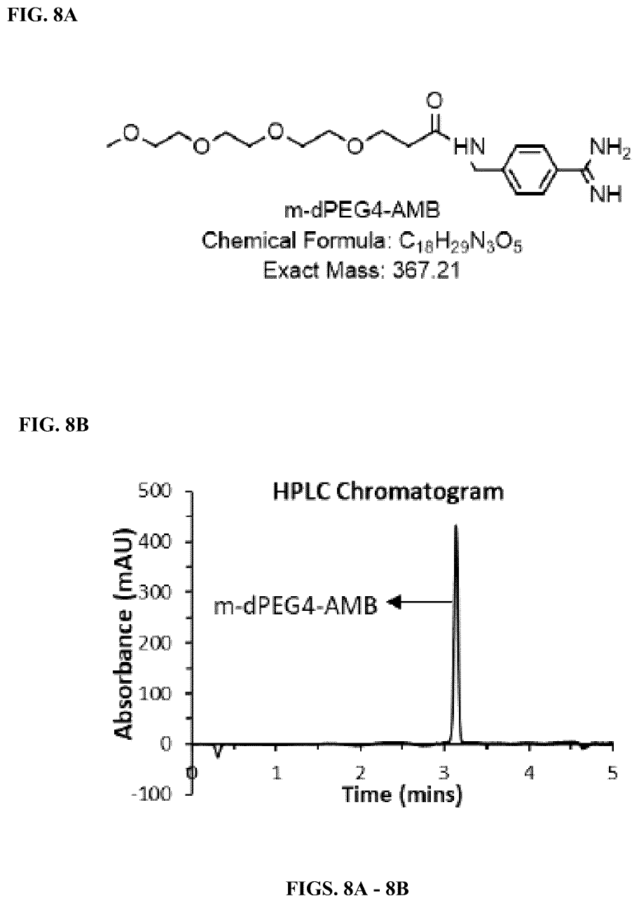

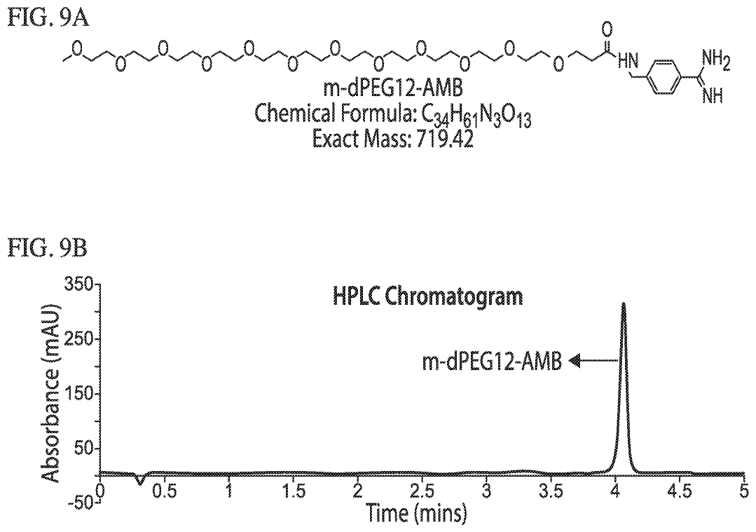

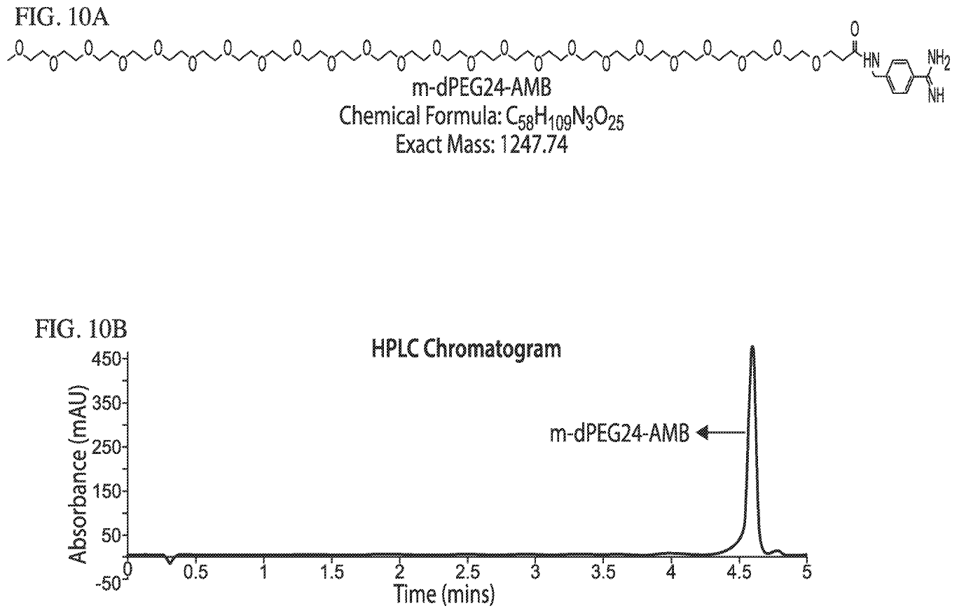

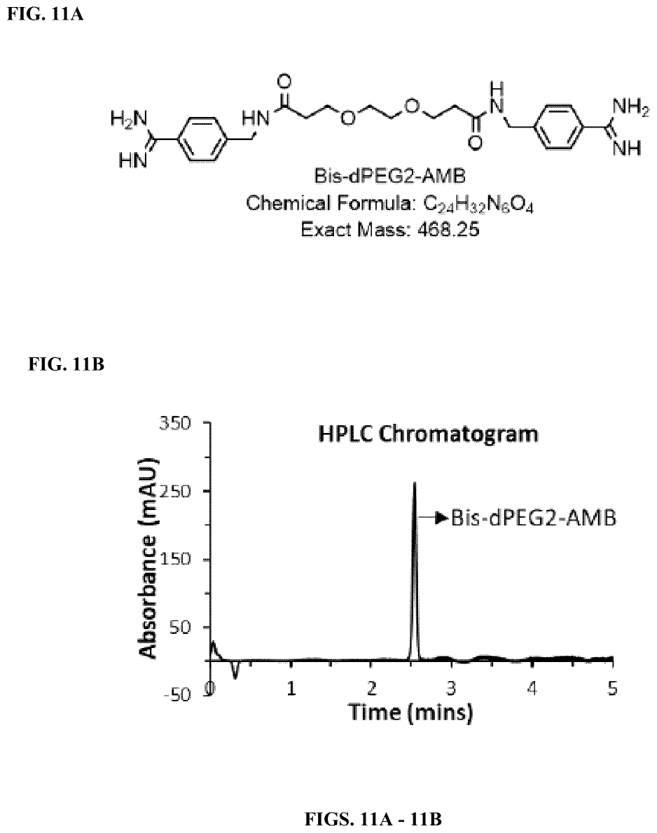

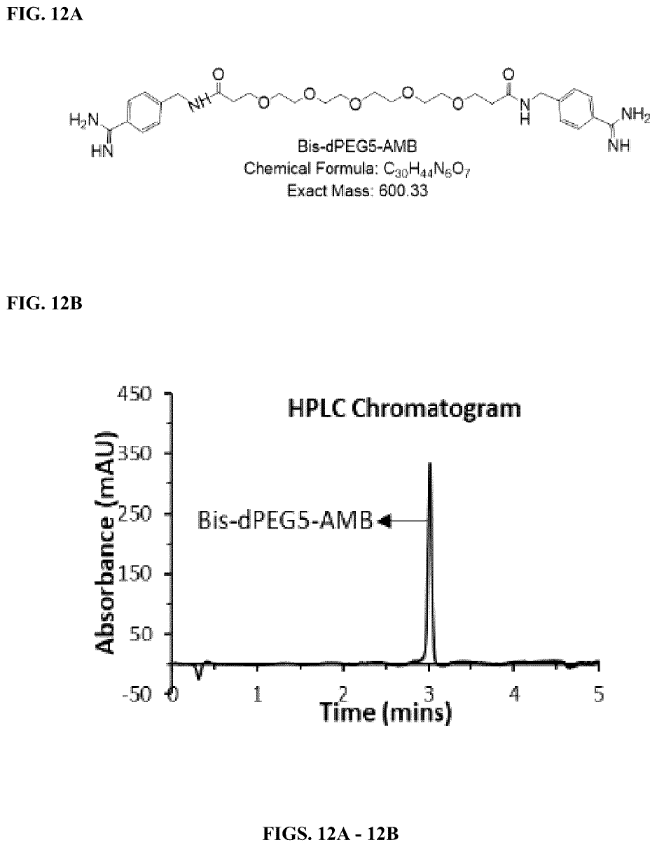

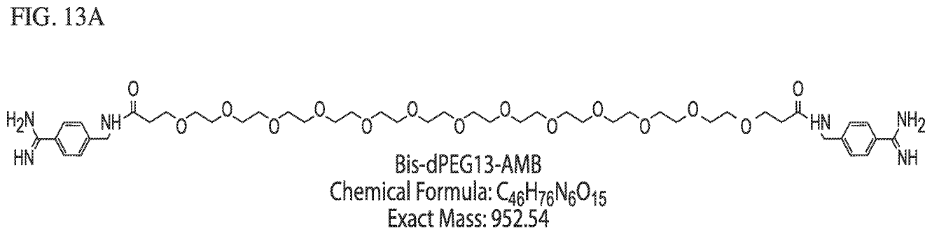

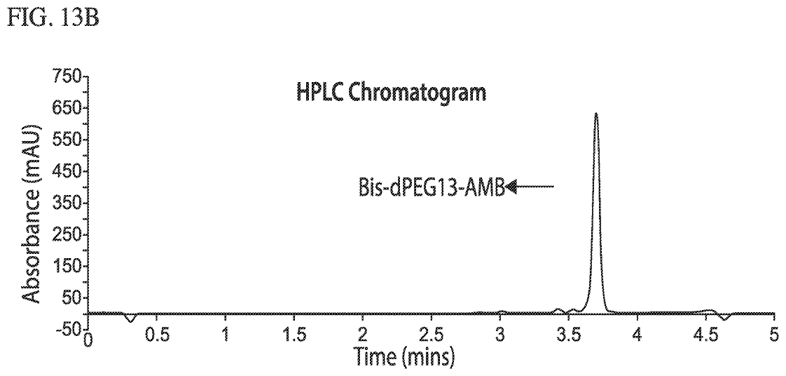

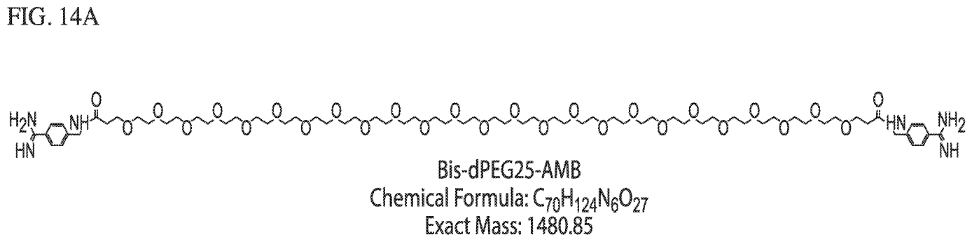

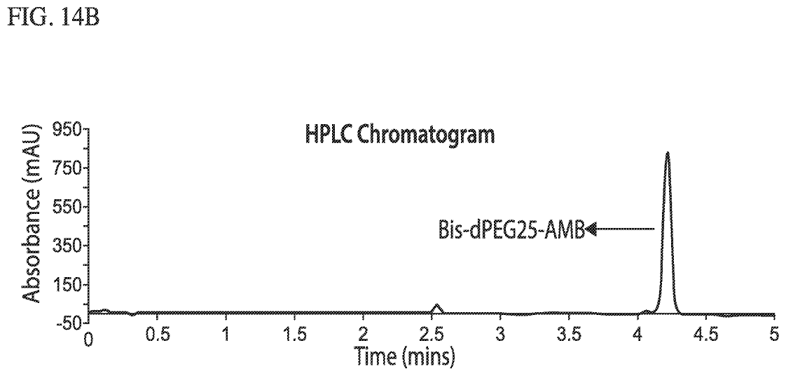

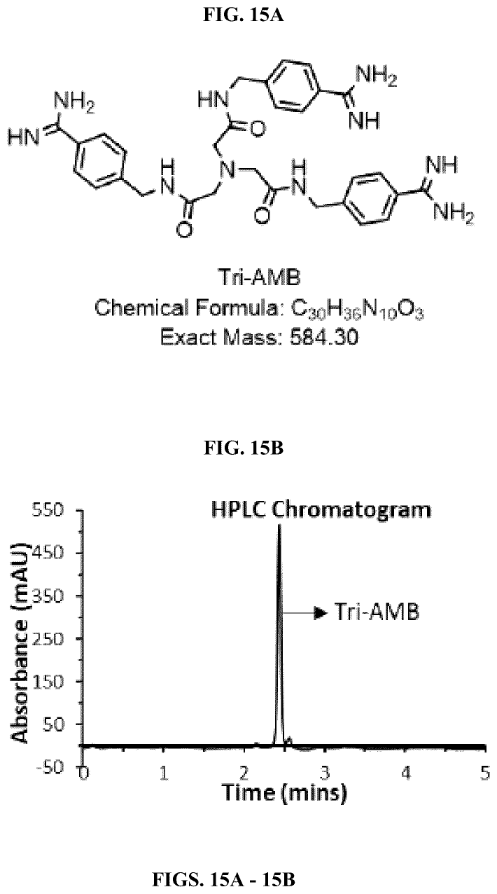

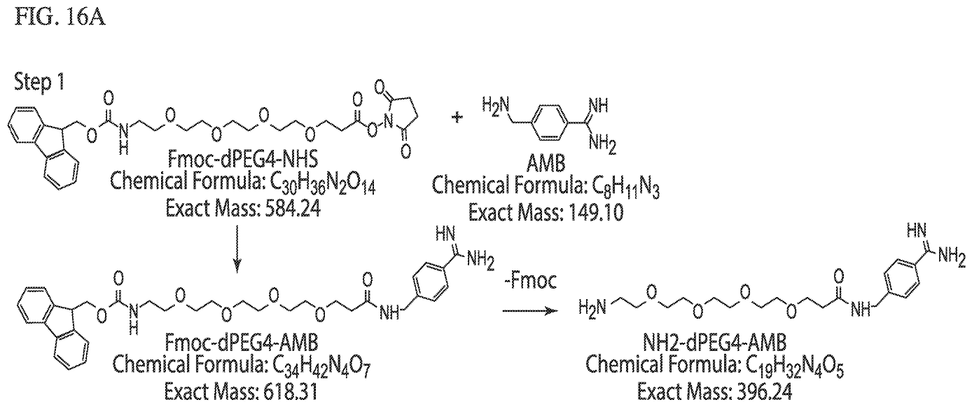

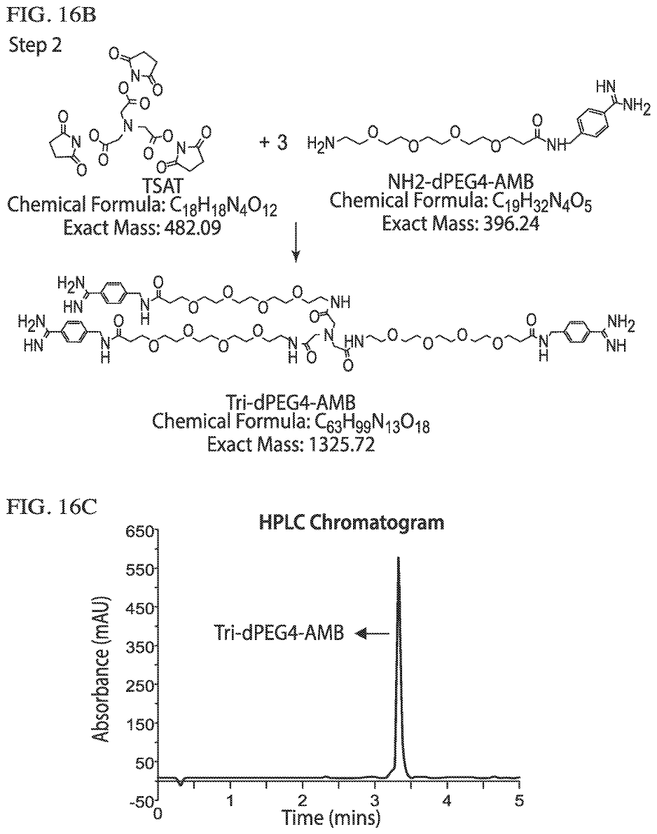

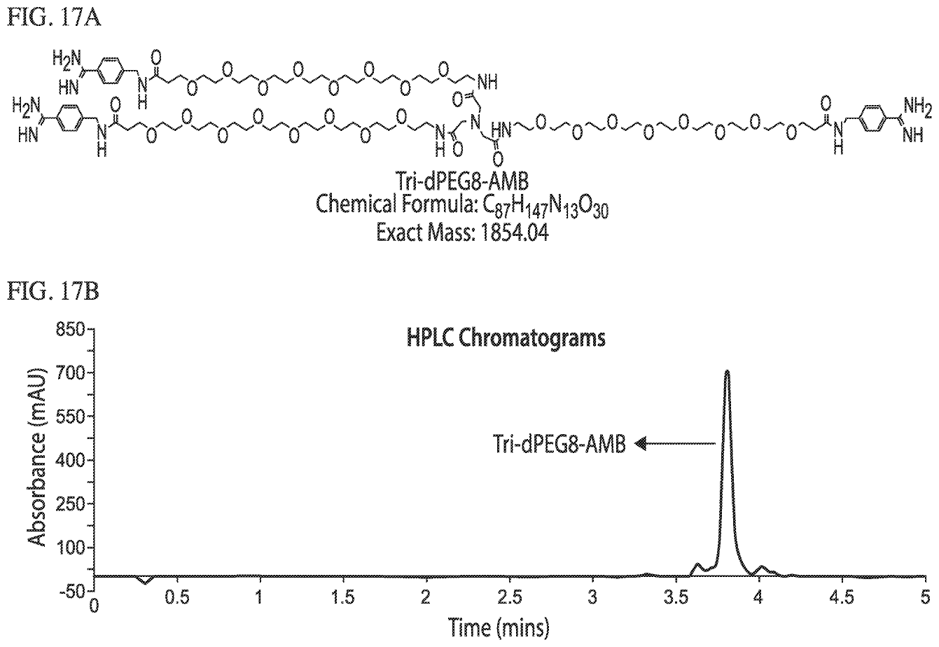

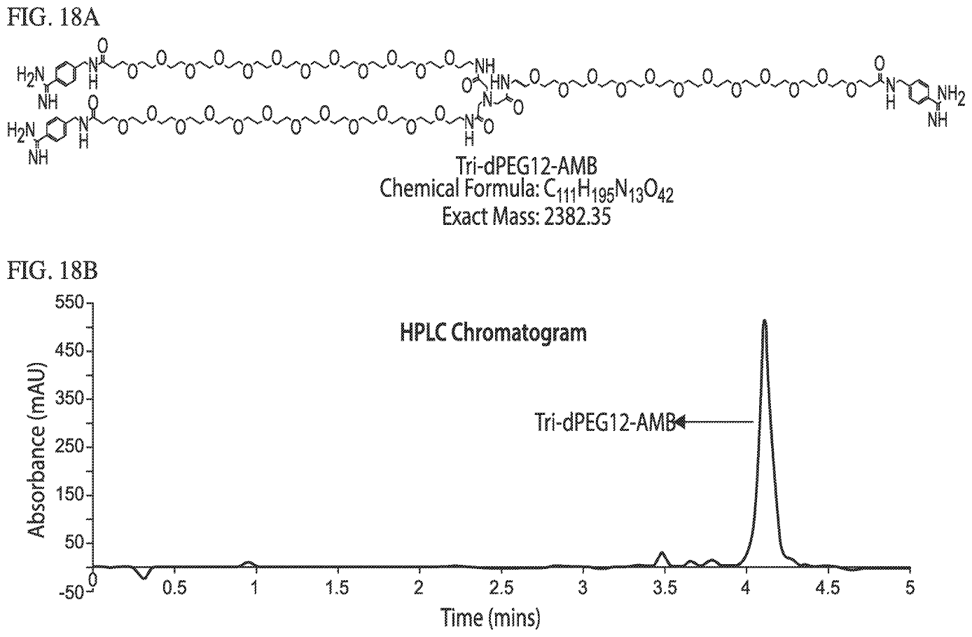

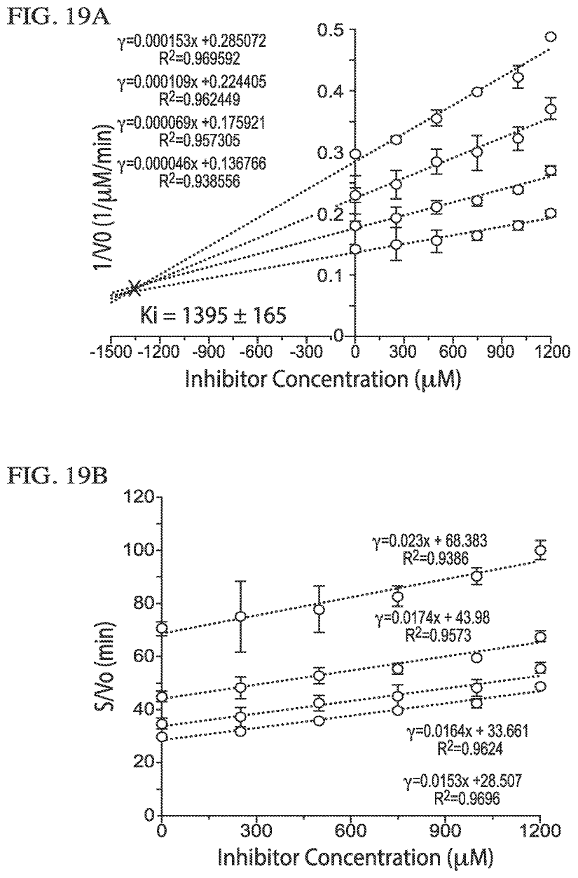

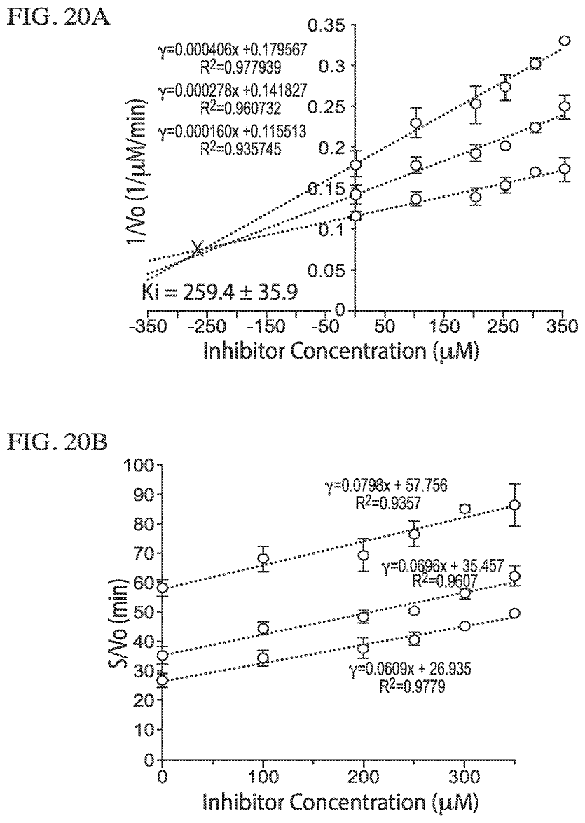

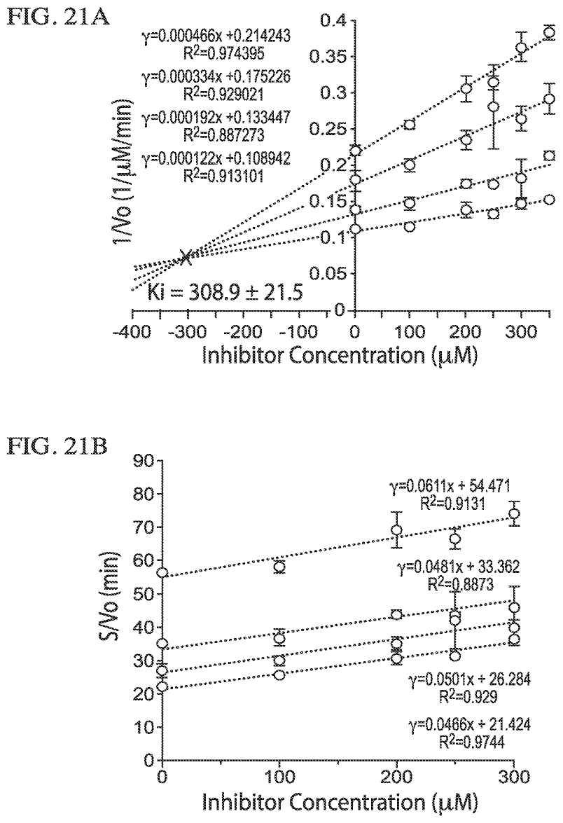

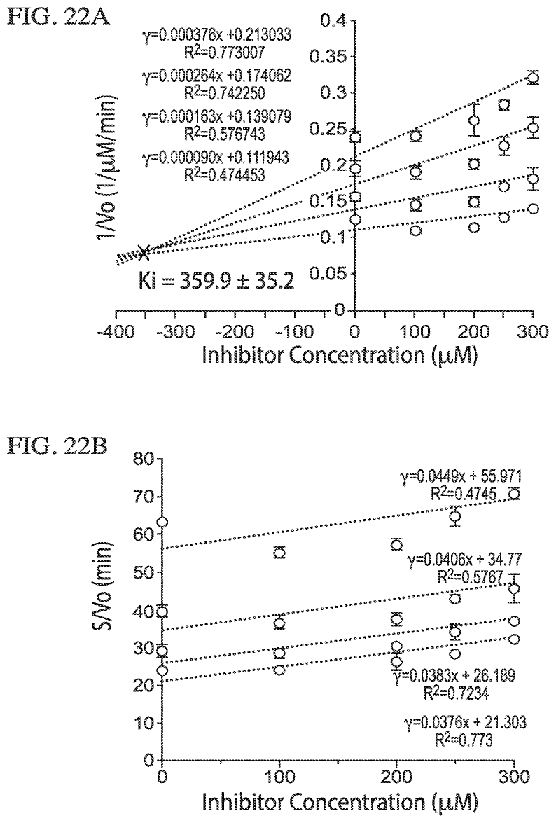

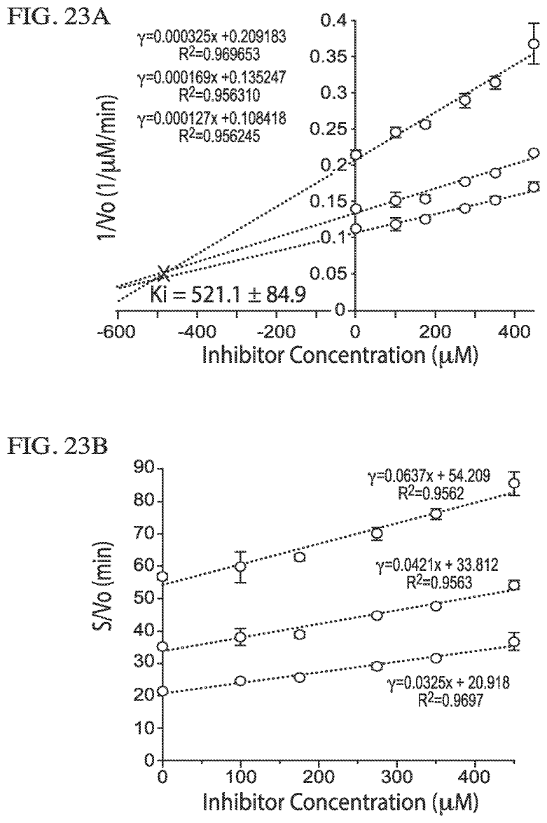

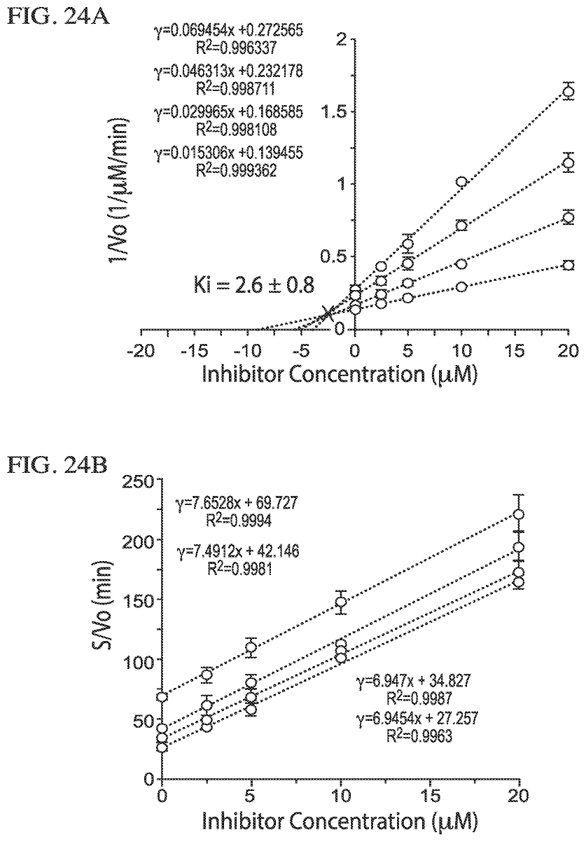

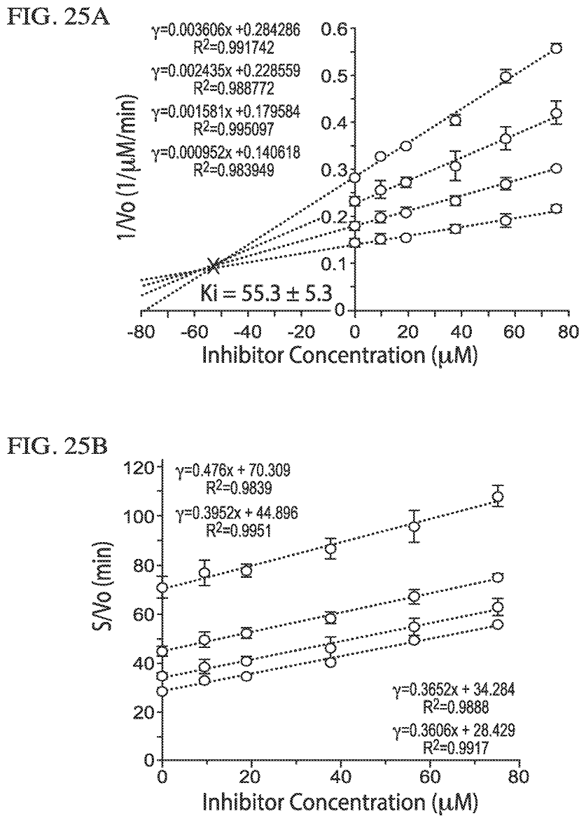

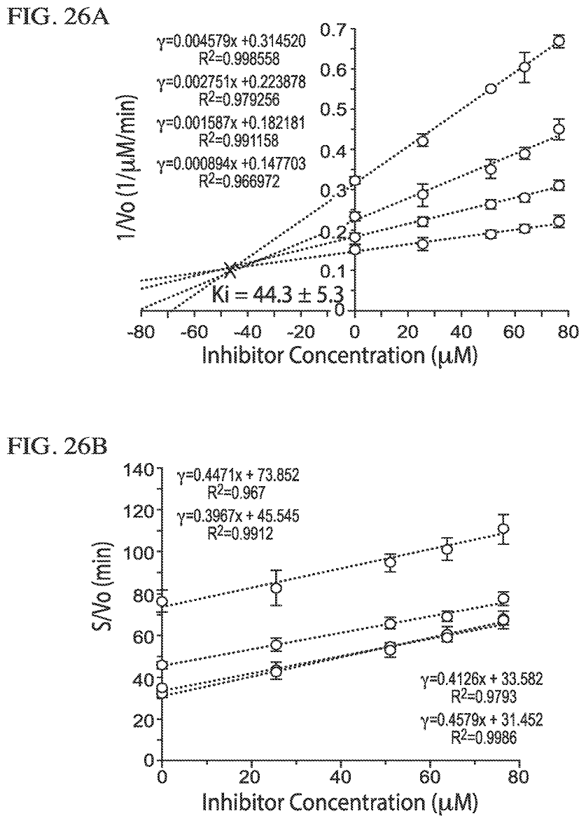

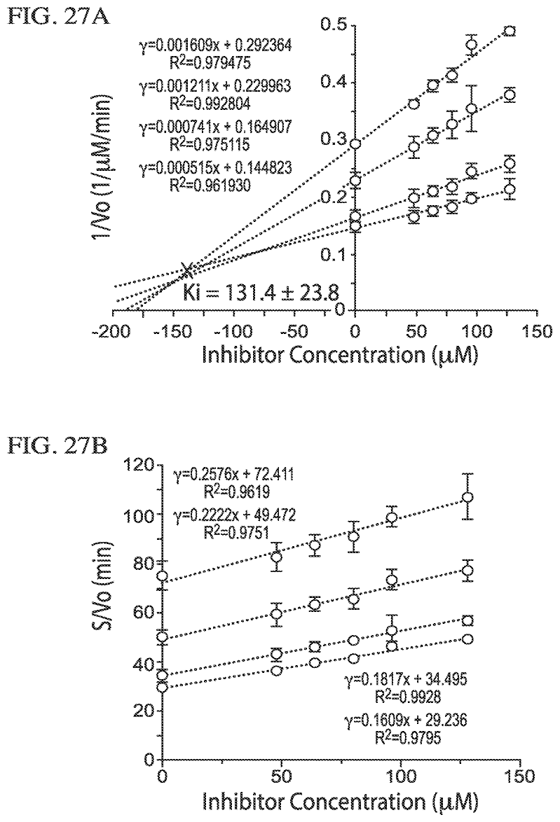

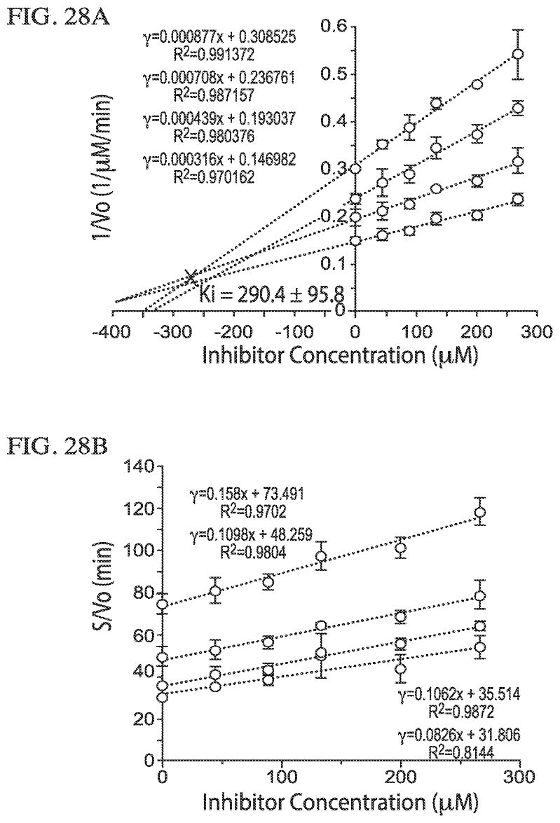

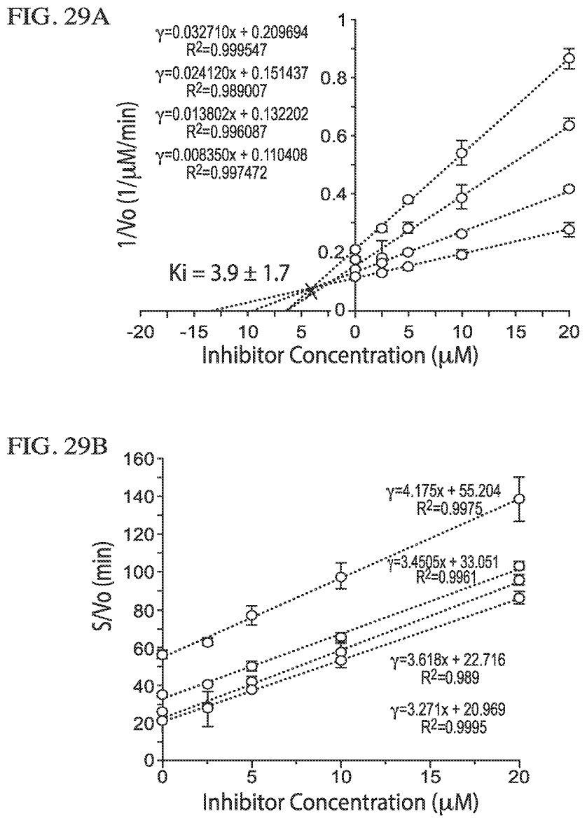

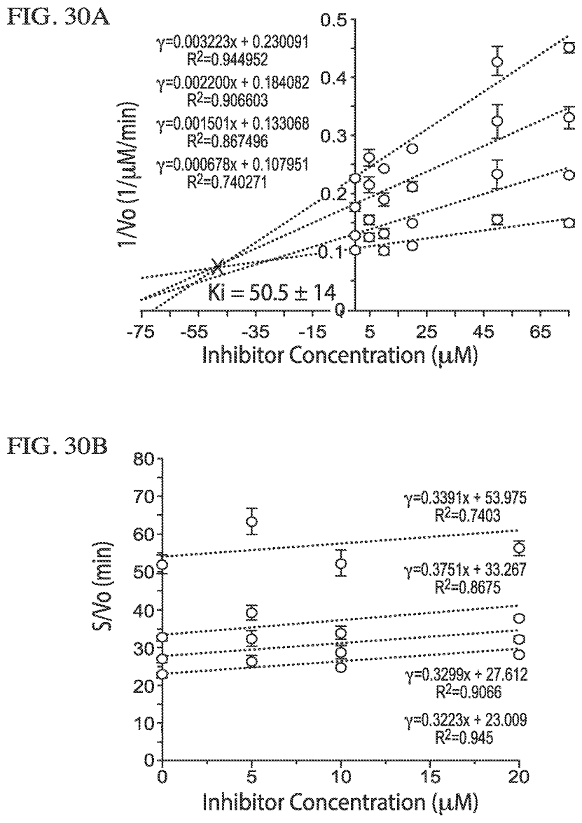

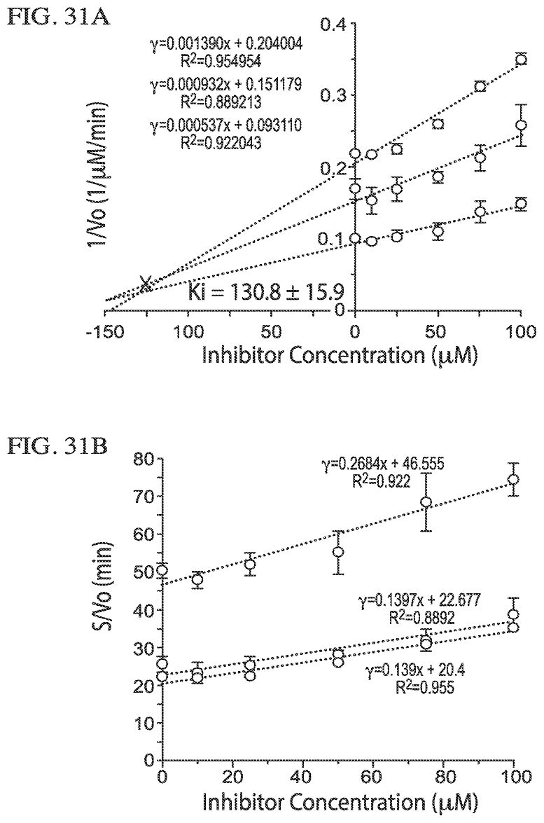

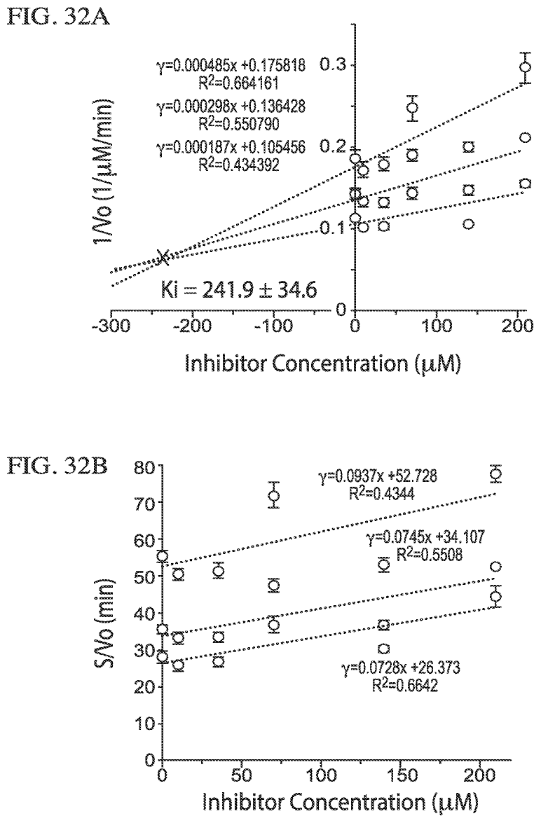

Monovalent (m-dPEGx-AMB; x=2, 4, 12, and 24), bivalent (Bis-dPEGx-AMB; x=2, 5, 13, and 25) and trivalent benzamidine (Tri-dPEGx-AMB; x=0, 4, 8, and 12) inhibitors were synthesized using 4-aminomethyl benzamidine (AMB) and PEG linkers via amine reactive n-Hydroxysuccinimide (NHS) chemistry. For trivalent inhibitor synthesis, a trivalent core TSAT was used (FIG. 4A). The reactions were performed in 0.01 M Phosphate Buffer Saline (PBS), pH 7.4 at room temperature (RT) and the inhibitors were purified using reverse phase High Performance Liquid Chromatography (RP-HPLC) and confirmed by mass spectrometry. HPLC Chromatogram and mass spectrum for Bis-dPEG2-AMB are shown as exemplary data in FIGS. 4B and 4C (see FIGS. 7A-30B for additional synthesis details).

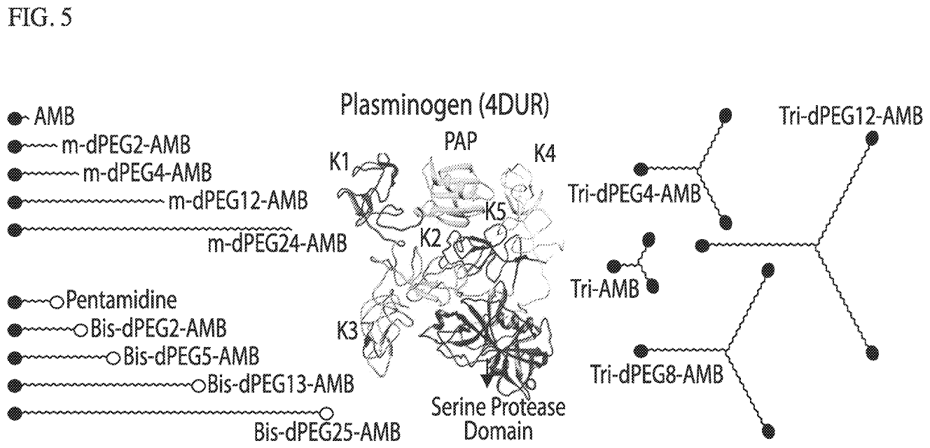

Inhibition assays were performed on all synthesized multivalent affinity molecules in addition to free AMB and Pentamidine. All multivalent affinity molecules are shown to scale relative to plasminogen (PDB ID: 4DUR) in FIG. 5 and the separation lengths are reported in Table 2. Inhibition constants (Ki) were determined using a chromogenic substrate (Chromogenix S-2251: H-D-Val-Leu-Lys-pNA·2HCl) specific for plasmin at a fixed concentration of plasmin (42.5 nM) over a range of substrate (100-500 μM) and multivalent affinity molecules concentrations (0-1,200 μM). Ki values were calculated for all multivalent affinity molecules via Dixon Plot analysis using the negative x-intersection point. Bis-dPEG2-AMB was included as exemplary data with additional Dixon Plots provided as the Appendices. A smaller Ki value indicates a higher degree of inhibition. Cornish-Bowden graphs (S/Vo vs I) were also plotted to verify the type of inhibition, i.e., competitive, uncompetitive, non-competitive, or mixed. The inhibition was determined to be purely competitive for all multivalent benzamidine affinity molecules as indicated by parallel lines on the Cornish-Bowden plots (Bis-dPEG2-AMB, and additional inhibitors shown in the Appendices).

TABLE 2
Plasmin inhibition by homo-monovalent, bivalent and
trivalent benzamidine inhibitors: inhibition constants
(Ki), rp, rp/n values and separation lengths.

| | | | | | Length |
|---|---|---|---|---|---|
| | Inhibitors | Kᵢ (μM) | rp^{[a]} | rp/n^{[b]} | (nm)^{[c]} |
| Monovalent | AMB | 1,395 ± 165.8 | — | — | 1.09 |
| (n = 1) | | | | | |
| | m-dPEG2- | 259.4 ± 35.9 | 5.4 | 5.4 | 2.13 |
| | AMB | | | | |
| | m-dPEG4- | 308.9 ± 21.5 | 4.5 | 4.5 | 2.91 |
| | AMB | | | | |
| | m-dPEG12- | 359.9 ± 35.2 | 3.9 | 3.9 | 6.19 |
| | AMB | | | | |
| | m-dPEG24- | 521.1 ± 84.9 | 2.7 | 2.7 | 11.03 |
| | AMB | | | | |
| Bivalent | Pentamidine | 2.1 ± 0.8 | 664.3 | 332.1 | 2.37 |
| (n = 2) | Bis-dPEG2- | 55.3 ± 5.3 | 25.2 | 12.6 | 3.34 |
| | AMB | | | | |
| | Bis-dPEG5- | 44.3 ± 5.3 | 31.5 | 15.7 | 4.48 |
| | AMB | | | | |
| | Bis-dPEG13- | 131.4 ± 23.8 | 10.6 | 5.3 | 7.64 |
| | AMB | | | | |
| | Bis-dPEG25- | 290.4 ± 95.8 | 4.8 | 2.4 | 12.39 |
| | AMB | | | | |
| Trivalent | Tri-AMB | 3.9 ± 1.7 | 357.7 | 119.2 | 2.25 |
| (n = 3) | Tri-dPEG4- | 50.5 ± 14 | 27.6 | 9.2 | 5.77 |
| | AMB | | | | |
| | Tri-dPEG8- | 130.8 ± 15.9 | 10.7 | 3.6 | 8.66 |
| | AMB | | | | |
| | Tri-dPEG12- | 241.9 ± 34.6 | 5.8 | 1.9 | 11.40 |
| | AMB | | | | |

^{[a]}rp: relative potency = Ki^{AMB}/Ki^{multi}.
^{[b]}rp/n: relative potency/number of benzamidine units.
^{[c]}calculated planar separation lengths between benzamidine moieties measured end to end using ChemDraw (Version 19.0.1.)

Ki values of mono-, bi-, and trivalent affinity molecules ranged from 259.4-1,395 μM, 2.1-290.4 μM, and 3.9-241.9 μM, respectively (Table 2). Parameters used to quantify multivalency, namely, relative potency (rp), and relative potency per unit (rp/n) were also computed. To evaluate the strength of the multivalent affinity molecules as compared to the monovalent affinity molecules, rp was used and is a ratio of Ki^{mono} to Ki^{multi} Ki of monovalent AMB was used for this calculation since AMB was utilized for synthesis of the multivalent affinity molecules. While rp>1 suggests that the multivalent affinity molecule is stronger, it does not take into account the increased concentration associated with greater than monovalent affinity molecules. To better evaluate multivalency effects, rp/n was also computed to determine the benefit of linking multiple affinity moieties together. A rp/n value >1 indicates that the potency of each affinity moiety in the multivalent system is stronger than the monovalent affinity molecule. If rp/n=1, it demonstrates that there is no benefit of linking affinity moieties together and it is equivalent to having “n” number of monovalent affinity moieties in solution. Finally, if rp/n<1, linking affinity moieties together is detrimental. The rp and rp/n values across valencies along with the planar separation lengths between benzamidine affinity moieties are summarized in Table 2.

All synthesized monovalent benzamidine affinity molecules were more potent than AMB with rp and rp/n values >1. This was potentially because the change in substituent group of the aromatic ring of benzamidine alters hydrophobic interactions between benzamidine and plasmin thus, effecting inhibition. All synthesized bivalent and trivalent affinity molecules exhibited beneficial multivalent effects with rp/n values >1. The rp/n values for bivalent and trivalent affinity molecules ranged from 2.4 to 332.1 and 1.9 to 119.2 respectively. This demonstrates that the potency of each benzamidine in these multivalent affinity molecules was at least 2-fold stronger than monovalent AMB and therefore, it is beneficial to link benzamidine affinity moieties together with a shared scaffold. Pentamidine, the shortest bivalent multivalent affinity molecule was the strongest multivalent plasmin inhibitor with a Ki value of 2.1±0.8 μM, rp of 664.3 an rp/n of much greater than 1 (332.1) comparable to the shortest trivalent affinity molecule Tri-AMB of Ki 3.9±1.7 μM.

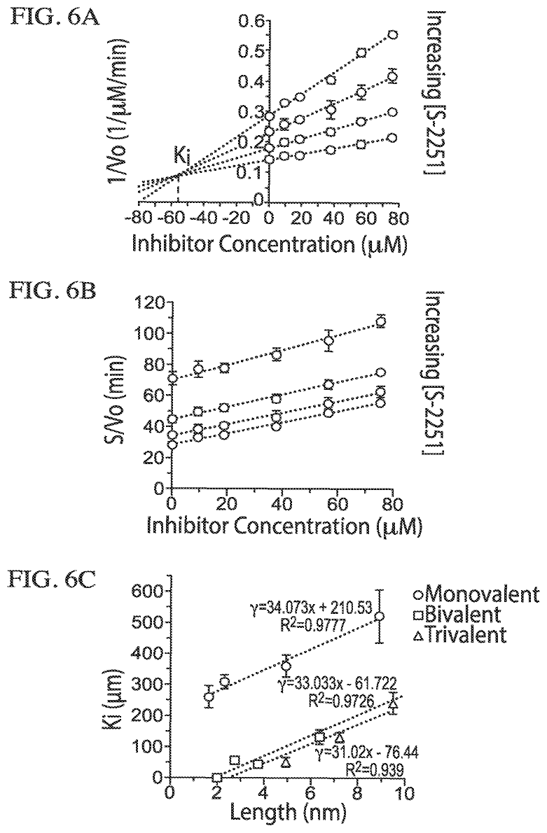

When Ki values of these multivalent affinity molecules were plotted against their planar separation lengths between two benzamidines, it was observed that Ki increased with length indicating weaker inhibition with longer linker lengths (FIG. 6C). This was expected as PEG is a flexible linker, the longer it is, the more are the conformational states it can occupy resulting in a higher entropic penalty for binding and a more negative ΔS. This leads to less negative and therefore, less favorable ΔG value (Equation 1). Less favorable ΔG value in turn results in higher Ki value (Equation 2) leading to lower inhibition with longer and more flexible linkers.

ΔG=ΔH−TΔS (Equation 1)

Ki=e^{ΔG/RT} (Equation 2)

As depicted in FIG. 6C, not only did inhibition decrease with linker length but was linearly correlated (R²>0.93). Moreover, mono-, bi-, and trivalent affinity molecules had similar slopes indicating similar rate of increase in Ki (or decrease in inhibition) per increase in unit length across all multivalent affinity molecules irrespective of their valencies. Also, comparing a specific separation length across valencies demonstrated that higher valency resulted in a smaller Ki value, or stronger inhibition (Trivalent>Bivalent>Monovalent inhibition). This is because valency affects multivalent inhibition. Increase in valency indicated that the number of inhibitor affinity moieties increased which in turn increased the effective concentration of the affinity moiety in the vicinity of the enzyme and therefore, promoted stronger inhibition. Although, trivalent affinity molecules were stronger than bivalent affinity molecules, both exhibited similar Ki values. These results conclude that multivalent inhibition can be modulated by valency and linker length and the higher the valency and the shorter the linker length, resulted in stronger inhibition.

### Example 2

In this Example, the effect of valency and linker length on active site inhibition of plasmin was determined using tranexamic acid (TXA).

TXA is a clinically utilized inhibitor of plasmin that predominantly binds to LBS on plasmin and is a very weak active site inhibitor with a Ki of 21 mM. PAMAM (Polyamidoamine) dendrimers were used to link TXA affinity moieties. PAMAM dendrimers of generation 0 to 2 corresponding to valencies of 4 to 16 were used to synthesize multivalent TXA of valencies 4 (PAMAM 4-TXA), 8 (PAMAM 8-TXA) and 16 (PAMAM 16-TXA). These dendrimers were synthesized using PAMAM dendrimers and Fmoc-TXA in DMF at room temperature using 2-(1H-benzotriazol-1-yl)-1,1,3,3-tetramethyluronium hexafluorophosphate (HBTU), N,N-Diisopropylethylamine (DIEA) and Oxyma Pure. The dendrimer product was precipitated with cold ethyl ether and washed with excess ether. The Fmoc was deprotected using 20% piperidine in DMF. The product was again precipitated with diethyl ether and washed with excess ether. This precipitate was solubilized in water and was dialyzed using Slide-A-Lyzer dialysis cassette against deionized water having 2.5 kDa MWCO to separate out the by-products from the conjugated dendrimer.²¹ Finally, the product of intended valency was purified using HPLC semi-preparative Thermo Hypersil GOLD C18 column (5 um, 250×10 mm) on a gradient of water and methanol with 0.10% trifluoroacetic acid and the masses were confirmed using mass spectrometry. In addition to these dendrimer-TXA inhibitors, bivalent Bis-TXA was also synthesized using Fmoc-Lys (Fmoc)-OH and Fmoc-TXA on a NovaPEG Rink amide resin via solid phase peptide synthesis (SPPS). The Fmoc was deprotected using 20% piperidine and the compound was cleaved from the resin using 95% TFA/2.5% TIS(Triisopropylsilane)/2.5% water. This was purified on HPLC using the method mentioned above and the mass was confirmed via mass spectrometry

TABLE 3
Plasmin inhibition by homo-multivalent tranexamic
acid (TXA) affinity molecules: inhibition constants
(Ki), rp, rp/n values and separation lengths.

| | | | | Length |
|---|---|---|---|---|
| Inhibitors | Kᵢ (μM) | rp^{[a]} | rp/n^{[b]} | (nm)^{[c]} |
| TXA | 21370 ± 3300 | — | — | 1.09 |
| Bis-TXA (n = 2) | 6401 ± 2035 | 3.3 | 1.7 | 2.1 |
| PAMAM 4 - TXA | 183 ± 27 | 117 | 29 | 4.12 |
| (n = 4) | | | | |
| PAMAM 8 - TXA | 2.5 ± 1.8 | 8548 | 1069 | 5.84 |
| (n = 8) | | | | |
| PAMAM 16 - TXA | 3.6 ± 3.0 | 5936 | 371 | 7.58 |
| (n = 16) | | | | |

^{[a]}rp: relative potency = Ki^{TXA}/Ki^{multi}.
^{[b]}rp/n: relative potency/number of TXA units.
^{[c]}calculated planar separation lengths between TXA moieties measured end to end using ChemDraw (Version 19.0.1.)

Inhibition assays were performed using methods described with benzamidine inhibitors. Human plasmin (42.5 nM) over a range of inhibitor concentrations (0-2 mM) and chromogenic substrate concentrations (Chromogenix S-2251 100-500 μM) were utilized for Inhibition assays. All the Ki values are shown in Table 3 along with rp, rp/n values and theoretical planar diameters. rp and rp/n values were calculated using TXA as the reference molecule as all other multivalent TXA affinity molecules were multivalent versions of TXA. From the inhibition assays, it was observed that TXA and Bis-TXA were weak active site inhibitors of plasmin with Ki values of 21 mM and 6.4 mM, respectively. As valency was increased from 1 to 8, multivalent TXA affinity molecules became stronger active site inhibitors of plasmin. PAMAM 8-TXA of valency 8 was the strongest multivalent TXA with a Ki of 2.47 μM. This multivalent TXA affinity molecule had a rp value of 8548 and rp/n value of 1069. These numbers indicate that PAMAM 8-TXA is over 8548-fold more potent than monovalent TXA and that each TXA moiety in this molecule is 1069-fold more potent than monovalent TXA. In addition, PAMAM 8-TXA was as strong as pentamidine, the strongest benzamidine inhibitor. The Ki value of PAMAM 16-TXA was 3.59 NM. This was comparable to PAMAM 8-TXA and indicates that increasing valency from 8 to 16 did not improve inhibition. This is probably due to valency and size acting as counteracting forces. Increase in valency was expected to increase the effective concentration of the inhibitor affinity moieties and an increase in the size of the inhibitor would decrease the effective concentration therefore opposing the effect of valency and not improving inhibition. However, PAMAM 16-TXA was a stronger inhibitor than all other multivalent TXA affinity molecules of valencies 1-4. Hence, the data from Table 3 indicates that TXA, a weak active site inhibitor that binds to the lysine binding sites on kringle domain of plasmin, can be converted to a strong active site inhibitor by means of multivalency.

### Example 3

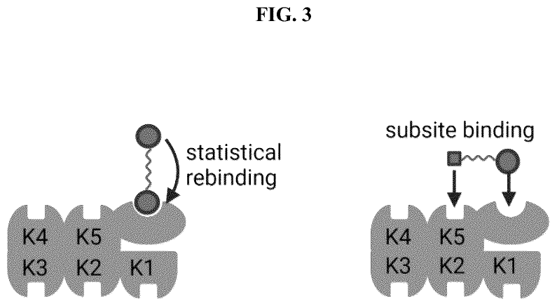

To determine whether stronger inhibition of plasmin could be achieved by leveraging the subsite binding effect of multivalency via benzamidine binding to active site of plasmin and TXA binding to the kringle domains (FIG. 3), hetero-bivalent inhibitors comprising both benzamidine and TXA were synthesized. Although TXA is a weak active site inhibitor of plasmin, it has a strong affinity for LBS on K1 domain (Ki=1.1 μM). TXA also binds weakly to the LBS sites on other kringle domains of plasmin with Ki of 750 NM. Hence, hetero-bivalent inhibitors can potentially be strong inhibitors of plasmin if benzamidine binds to the active site and TXA binds to the LBS on kringle domain specifically K1 on plasmin simultaneously.

Hetero-bivalent inhibitors of different PEG lengths were synthesized utilizing AMB, TXA and Fmoc-dPEGx-NHS esters. AMB was first reacted with Fmoc-dPEGx-NHS/TFP esters (x=4,8,12,36) in a mixture of DMF and PBS. The reaction crude was dried and the Fmoc was deprotected using 20% piperidine in DMF. The reaction crude was dried again and the product NH2-dPEGx-AMB was selectively solubilized in water and then was purified on HPLC using semi-preparative Thermo Hypersil GOLD C18 column (5 um, 250×10 mm) on a gradient of water and methanol with 0.10% trifluoroacetic acid. The masses were confirmed using mass spectrometry (see Supporting Information). The NH2-dPEGx-AMB were then reacted with Fmoc-TXA in DMF at room temperature using HBTU/DIEA. Finally, Fmoc was deprotected using 20% piperidine and the product was purified on HPLC according to the method described above. The final product masses were confirmed with mass spectrometry to ensure the appropriate molecular masses.

Inhibition assays were performed using methods described above with benzamidine and TXA inhibitors. Human plasmin (42.5 nM) over a range of inhibitor concentrations (0-500 μM) and chromogenic substrate concentrations (Chromogenix S-2251 100-500 μM) were utilized for Inhibition assays. All the Ki values are shown in Table 5 along with rp values and theoretical planar diameters. rp values were calculated using both TXA and AMB as the reference molecules to determine how potent these inhibitors are relative to TXA and AMB. From Table 3, it is evident that all the synthesized hetero-bivalent inhibitors are more potent that the monovalent versions of AMB and TXA. These inhibitors are at least 10-fold more potent than monovalent AMB and over 100-fold more potent than monovalent TXA. It is also observed that even in hetero-bivalent linkers, inhibition decreases with increase in linker length owing to entropic penalty. In addition, AMB-dPEG4-TXA is over 7-fold stronger than m-dPEG4-AMB indicating that the hetero-bivalent inhibitors portray a multivalent effect and linking AMB and TXA together is beneficial. Heterobifunctional linkers were weaker inhibitors as linker length increased up until additional subsite binding locations became accessible due to a linker being sufficiently long. This is illustrated by the results showing TXA-PEG12-AMB was 207 μM vs the longer TXA-PEG36-AMB molecule having more inhibition (tighter binding) of 75 μM. This supports the concept that linkers (or linker lengths) can be designed to allow for specific subsite binding to drive specificity and effect binding affinity.

TABLE 4
Plasmin inhibition by hetero-bivalent inhibitors: inhibition
constants (Ki), rp, rp/n values and separation lengths.

| | | | | Length |
|---|---|---|---|---|
| Inhibitors | Kᵢ (μM) | rp1^{[a]} | rp2^{[b]} | (nm)^{[c]} |
| AMB | 1395 ± 166 | — | — | 1.09 |
| TXA | 21370 ± 3300 | — | — | 1.09 |
| TXA-dPEG4- | 42 ± 12.4 | 33 | 509 | 3.95 |
| AMB | | | | |
| TXA-dPEG8- | 99 ± 0.9 | 14 | 216 | 5.52 |
| AMB | | | | |
| TXA-dPEG12- | 207 ± 4 | 7 | 103 | 7.11 |
| AMB | | | | |
| TXA-dPEG36- | 75.4 ± 4.3 | 19 | 285 | 16.59 |
| AMB | | | | |
| EACA | 71150 ± 25540 | — | — | 1.18 |
| EACA-dPEG4- | 233 ± 5 | 6 | 305 | 4.08 |
| AMB | | | | |

^{[a]}rp1: relative potency = Ki^{AMB}/Ki^{multi}.
^{[b]}rp2: relative potency = Ki^{TXA} or EACA/Ki^{multi}
^{[c]}calculated planar separation lengths between TXA moieties measured end to end using ChemDraw (Version 19.0.1.)

Three different classes of multivalent affinity molecules were synthesized for inhibiting plasmin leveraging the principles of multivalency. With benzamidine affinity molecules that were monovalent, bivalent and trivalent, it was observed that valency enhanced inhibition and long linker lengths decreased inhibition due to entropic effects. With multivalent TXA dendrimers, it was determined that TXA, which is a weak active site inhibitor, can be transformed into a strong active site inhibitor utilizing multivalency. It was also observed that inhibition with the PAMAM-TXA dendrimers increased with valency until the size of the molecule probably became a counteracting force that potentially reduced effective concentration. Strong plasmin inhibition can also be achieved with hetero-multivalent affinity molecules that have different affinity moieties that can achieve stronger inhibition by targeting different sites on the enzyme. Hence, multivalency is a unique and highly effective strategy that can be used to synthesize strong inhibitors. To achieve desirable inhibition, the valency and linker length of the multivalent affinity molecule can be modulated.

### Example 4

Benzamidine and its derivatives are known to inhibit thrombin which is also a serine protease. In this example, commercially available inhibitors shown in Table 1, Tri-AMB and all hetero-multivalent inhibitors were tested for their thrombin inhibition. Inhibition assays were performed using a fixed thrombin concentration of 0.25 U/mL in PBS pH 7.4 over a range of Thrombin Substrate III (fluorogenic) concentrations of 20-50 μM at 10% DMSO. Dixon plots analysis was performed to obtain Ki values that are shown in Table 5. All commercial monovalent benzamidine derivatives except for benzamidine exhibit the same inhibition trend as seen with plasmin. Pentamidine and Tri-AMB are slightly weaker thrombin inhibitors compared to plasmin and pentamidine is slightly stronger thrombin inhibitor than Tri-AMB as seen with plasmin. It was also interesting to see that all hetero-multivalent inhibitors were stronger inhibitors of thrombin than plasmin despite thrombin not having kringle domains. However, as thrombin does not have any kringle domains, weaker inhibition with longer linker lengths was observed as there was no subsite binding.

TABLE 5
Thrombin inhibition by various monovalent and multivalent
benzamidine, TXA and EACA derived multivalent affinity
molecules represented by inhibition constants (Ki).

| Inhibitors | Kᵢ (μM) |
|---|---|
| Benzamidine | 63 ± 11 |
| 4-amino benzamidine | 43 ± 13 |
| 4-aminomethyl | 344 ± 33 |
| benzamidine | |
| 4-carboxy benzamidine | 151 ± 4 |
| Pentamidine | 4.5 ± 2.3 |
| Tri-AMB | 10.6 ± 2 |
| TXA | 210,382 ± 41,400 |
| TXA-dPEG4-AMB | 14.5 ± 6.7 |
| TXA-dPEG12-AMB | 46 ± 1.5 |
| TXA-dPEG36-AMB | 78 ± 1.5 |
| EACA | >10,000 |
| EACA-dPEG4-AMB | 5.6 ± 0.2 |

### Example 5

Benzamidine and its derivatives are known to inhibit tPA which is also a serine protease that has 2 kringle domains. In this example, commercially available inhibitors shown in Table 1, Tri-AMB, TXA-PEG4-AMB and TXA-PEG12-AMB inhibitors were tested for their tPA inhibition. Inhibition assays were performed using a fixed tPA concentration of 75 nM in PBS pH 7.4 over a range of Chromogenic tPA Substrate (S-2288) concentrations of 100-500 μM. Dixon plots analysis was performed to obtain Ki values that are shown in Table 6. All commercial monovalent benzamidine derivatives are weaker tPA inhibitors compared to plasmin but exhibited similar inhibition trend. Pentamidine and Tri-AMB are also weaker tPA inhibitors compared to plasmin and pentamidine is a stronger inhibitor than Tri-AMB. It was also interesting to see that TXA-PEG12-AMB is a stronger tPA inhibitor than TXA-PEG4-AMB.

TABLE 6
tPA inhibition by monovalent and multivalent benzamidine,
TXA and EACA derived multivalent affinity molecules
represented by inhibition constants (Ki).

| Inhibitors | Kᵢ (μM) |
|---|---|
| Benzamidine | 438 ± 2.4 |
| 4-amino benzamidine | 148 ± 0.9 |
| 4-aminomethyl | 5209 ± 161 |
| benzamidine | |
| Pentamidine | 48 ± 12 |
| Tri-AMB | 112 ± 21 |
| TXA | 122850 ± 33950 |
| TXA-dPEG4-AMB | 230 ± 30 |
| TXA-dPEG12-AMB | 128 ± 11 |

### Example 6

In this example, homo-multivalent and hetero multivalent micelles were synthesized to determine their plasmin inhibition. Homo multivalent micelles (Methoxy Amine Benz) and hetero-multivalent micelles (Methoxy Benz TXA) were synthesized along with Methoxy Bulk and Methoxy Amine micelles as controls. After weighing and mixing the appropriate lipids amounts in chloroform, a lipid film was made by slowly evaporating chloroform while rotating the vial gently under air. The dried lipid film was placed in a desiccator under vacuum overnight. The lipid film was rehydrated using 2 mL PBS and was sonicated for 2 mins while heating at 60° C. to obtain more rapid and less polydisperse micelle formation by self-assembly. Inhibition assays were performed using methods described above with benzamidine and TXA inhibitors. Human plasmin (42.5 nM) over a range of inhibitor concentrations (0-500 μM) and chromogenic substrate concentrations (Chromogenix S-2251 100-500 μM) were utilized for Inhibition assays. All the Ki values are shown in Table 7. Hetero-multivalent Methoxy Benz TXA micelles that had both Benzamidine and TXA were the strongest inhibitors with a Ki of 35±1.4 μM followed by homo-multivalent Methoxy Amine Benz micelles. These micelles were stronger than Methoxy Amine and Methoxy Bulk control micelles that exhibited non-specific inhibition.

TABLE 7
Plasmin inhibition by DSPE-PEG2000 micelles
represented by inhibition constants (Ki).

| Inhibitors | |
|---|---|
| (Methoxy:Amine:Benz:TXA) | Kᵢ (μM) |
| Methoxy Bulk (100:0:0:0) | 346 ± 0.12 |
| Methoxy Amine (75:25:0:0) | 101 ± 13 |
| Methoxy Amine Benz (50:25:25:0) | 89 ± 2 |
| Methoxy Benz TXA (50:0:25:25) | 35 ± 1.4 |

Applying the principles of multivalency for therapeutic applications can be advantageous as it gives the ability to tune overall binding avidity simply by modifying the valency (“n”, number of affinity ligands or inhibitor moieties) of the multivalent affinity molecule. In addition to valency, linker length, flexibility of linker, shape and spatial orientation are other parameters that affect multivalency. Unlike monovalent interactions that have only two binding modes, binding or no binding, multivalent interactions can provide many different binding modes ranging from all affinity moieties being bound, some bound and some unbound, and all unbound. Enhanced binding through multivalency can be achieved by four different mechanisms: (a) statistical rebinding: close proximity of multiple moieties promotes rebinding; (b) chelate effect: multiple moieties simultaneously bind to multiple active sites; (c) subsite binding: interactions with both active and non-active domains or multiple interactions with non-active domains; (d) clustering: molecular interaction across multiple enzymes.

1. A multivalent affinity molecule comprising:
two affinity moieties to about 100 affinity moieties; and
a scaffold,
wherein the two affinity moieties to about 100 affinity moieties are covalently coupled to the scaffold.

2. The multivalent affinity molecule of claim 1, wherein the two affinity moieties to about 100 affinity moieties are the same affinity moieties.

3. The multivalent affinity molecule of claim 1, wherein at least one of the two affinity moieties to about 100 affinity moieties are different affinity moieties.

4. The multivalent affinity molecule of claim 3, wherein at least one of the affinity moieties binds a first domain of a target molecule and at least another affinity moieties binds a second domain of the target molecule.

5. The multivalent affinity molecule of claim 1, wherein the scaffold is a flexible linker, a rigid linker, a biodegradable polymer, a non-biodegradable polymer, a dendrimer, a micelle, a liposome, an organic particle, an inorganic particle, and combinations thereof.

6. The multivalent affinity molecule of claim 1, wherein the affinity moieties are selected from the group consisting of a small molecule compound, a protein, a peptide, and combinations thereof.

7. A method of treating a bleeding disorder in a subject in need thereof, the method comprising: administering to the subject in need thereof a multivalent affinity molecule comprising: two affinity moieties to about 100 affinity moieties; and a scaffold, wherein the two affinity moieties to about 100 affinity moieties are covalently coupled to the scaffold; and wherein the subject in need thereof has or is suspected of having hyperfibrinolysis-associated bleeding during surgery, trauma, disseminated intravascular coagulation, hemophilia, menorrhagia, and combinations thereof.

8. The method of claim 7, the affinity moiety is selected from a small molecule compound, a protein, a peptide, and combinations thereof.

9. The method of claim 8, wherein the affinity moiety is selected from the group consisting of a cyclohexanone, a cyclohexane, a quinidine, an amidine, a peptide, a tripeptide that comprises a nitrile warhead, a cyclic peptidomimetic, and combinations thereof.

10. The method of claim 9, wherein the affinity moiety is selected from the group consisting of benzamidine, a benzamidine derivative, benzylamine, tranexamic acid (TXA), e-aminocaproic acid (EACA), lysine, and combinations thereof.

11.-17. (canceled)

18. A method for prophylaxis of a bleeding disorder in a subject in need thereof, the method comprising: administering to the subject in need thereof a multivalent affinity molecule comprising: two affinity moieties to about 100 affinity moieties; and a scaffold, wherein the two affinity moieties to about 100 affinity moieties are covalently coupled to the scaffold.

19. The method of claim 18, the affinity moiety is selected from a small molecule compound, a protein, a peptide, and combinations thereof.

20. The method of claim 19, wherein the affinity moiety is selected from the group consisting of a cyclohexanone, a cyclohexane, a quinidine, an amidine, a peptide, a tripeptide that comprises a nitrile warhead, a cyclic peptidomimetic, and combinations thereof.

21. The method of claim 20, wherein the plasmin inhibitor is selected from the group consisting of benzamidine, a benzamidine derivative, benzylamine, tranexamic acid (TXA), e-aminocaproic acid (EACA), lysine, and combinations thereof.

22.-27. (canceled)

28. The method of claim 7, wherein the two affinity moieties to about 100 affinity moieties are the same affinity moieties.

29. The method of claim 7, wherein at least one of the two affinity moieties to about 100 affinity moieties are different affinity moieties.

30. The method of claim 29, wherein at least one of the affinity moieties binds a first domain of a target molecule and at least another affinity moieties binds a second domain of the target molecule.

31. The method of claim 18, wherein the two affinity moieties to about 100 affinity moieties are the same affinity moieties.

32. The method of claim 18, wherein at least one of the two affinity moieties to about 100 affinity moieties are different affinity moieties.

33. The method of claim 32, wherein at least one of the affinity moieties binds a first domain of a target molecule and at least another affinity moieties binds a second domain of the target molecule.
